(19) United States
(12) Patent Application Publication
(10) Pub. No.: US 2021/0134434 A1
(43) Pub. Date: May 6, 2021

(54) System and Method for Improving Food Selections

(71) Applicant: American Heart Association, Inc., Dallas, TX (US)

(72) Inventors: Christopher Riley, Dallas, TX (US); David Woody, Austin, TX (US); Ann Bray, Prosper, TX (US); Taylor Crutsinger, Dallas, TX (US); Chirag Chhita, Lewisville, TX (US); Navneet Kumar Ramachandran, Puducherry (IN); Kristyna Aldrich, Frisco, TX (US); Noelle Hutchins Kelso, Madison, AL (US); Autumn Jones, Frisco, TX (US)

(21) Appl. No.: 17/090,023

(22) Filed: Nov. 5, 2020

Related U.S. Application Data
(60) Provisional application No. 62/930,910, filed on Nov. 5, 2019.

(51) Int. Cl. G16H 20/60; G16H 50/30

(52) U.S. Cl. CPC G16H 20/60; G16H 40/67; G16H 50/30

(57) ABSTRACT

A method and system for improving a user's food selections by comparing one or more attributes of a food selection, such as nutritional profile, to one or more subjective criteria, such as a user's medical conditions, food allergies, and preferences for certain types of foods (vegan or vegetarian), and/or objective criteria, such objective food scores for the food selection, to provide a user with information regarding the relative healthfulness of a particular item compared to another and to incentivize the user to select more healthful items. Alternative items of equal or better food score may be suggested. A user's selections and scores for those selections are tracked over time to allow for comparisons to the user's prior data and to data for other user's having one or more common attributes to provide a competitive incentive for improvements. Rewards may also be provided for improvements or positive selections.

### CROSS REFERENCE TO RELATED APPLICATION

This application claims the benefit of U.S. Provisional Application Ser. No. 62/930,910 filed on Nov. 5, 2019.

### BACKGROUND OF THE INVENTION

### 1. Field of the Invention

This invention relates to a system and method for improving a user's food selections by comparing one or more attributes of a food selection, such as nutritional profile or user preferences, to one or more subjective and/or objective criteria to provide a user with information regarding the relative healthfulness, production, and other criteria of a particular food item compared to another and to incentivize the user to select more healthful and relevant items.

### 2. Description of Related Art

A problem frequently encountered by consumers in shopping for food items is determining the healthiest option among many food product options. Packaged foods include a nutrition label including information regarding the number of calories and amounts of various components, such as protein, fats, sugars, per serving of the packaged food. However, consumers can have difficulty deciphering the information provided, particularly since there is a lack of uniformity in labeling regarding the number of servings in a package and portion size. Other problems frequently encountered by consumers is making healthy food selections within a pre-determined budget, avoiding impulse purchases of unhealthy food items, and healthy meal planning. Additionally, consumers are also interested in other information regarding food items, such as sustainability, how the food was raised, produced, processed, and/or packaged, whether production and packaging of the food item is environmentally friendly, whether the item is local and where the items originated. It is often difficult to find this information, particularly in a grocery setting and particularly in a way that allows the consumer to compare the information for different items.

There are several known methods for scoring food items based on health criteria and providing the scores to a consumer to aid in making healthy food selections. For example, U.S. Pat. No. 8,647,121 discloses using a combination of binary (yes/no) and non-binary (quantitative) scoring of food items based on a consumer's personal information (such as peanut allergies, which would result in a binary score of “no”) and nutritional information for the food (such as the amount of sodium in a particular food and whether the consumer has hypertension). The score can be presented to the consumer as a letter grade (A to F), a number of stars, a numerical score, or a relative rating (Very Good to Very Bad). As another example, U.S. Pat. No. 7,974,881 discloses using a scoring algorithm and a set of rating correlations, which are the same for different food types. Numerical values are assigned for various nutritional attributes of a food product and are summed to provide an overall score. For example, if the amount of saturated fat in a food item is less than or equal to 1 g, the food item is assigned a score of 0 for that attribute and if the amount of sodium for the food item is more than 120 mg but less than or equal to 240 mg, the food item is assigned a score of −1 for that attribute. Negative scores are used for generally unhealthy attributes (fat, sodium, added sugars) and positive scores are used for generally healthy attributes (fiber). Another example, implemented in Europe, is the NutriScore system. This system assigns a color-coded letter grade (a green A for healthies to a red E for unhealthiest) for food items based on the nutritional profile of the food. While these and other food scoring systems and methods are useful for consumers, there is still a need for a system and method that integrates food scoring with point-of-purchase suggestions for healthier alternative foods, meal planning, and helps meet a consumer's budget goals by coupling grocery store sales and coupons with healthy foods and meal planning. There is also a need for a system and method that provides a consumer with more useful information to track his/her progress in making healthier food selections and a scoring function to allow a consumer to better understand whether his/her overall food selections are healthy.

### SUMMARY OF THE INVENTION

A method and system according to a preferred embodiment of the invention allows a user to input certain personal survey data or information regarding the user, user's food preferences (which may include factors regarding food sustainability, environmental friendliness, and how the food was produced, processed, and/or packaged that are of interest to the consumer), and the user's personal health risk factors, which are used as criteria to rank a user's food selections for a shopping event (for example, the food items searched or placed in the user's cart during an online grocery purchase), as part of planning a shopping event (for example, the food items that are searched and added to an online shopping list to be taken to the store for in-person purchase), and/or to enhance a user's in-store or point-of-purchase experience. One or more attributes of available food selections, such as nutritional information, whether the selected food is organic, and price, are compared to one or more user criteria from the personal survey data and/or objective criteria (such as science-based criteria) to determine if the food selections meet the user's criteria and the items are ranked according to how well the item meets the criteria so the user may assess whether to purchase items in-store, at the point of purchase, or include the food items in a shopping cart or list. Preferably, the personal survey data includes information regarding the user's age, race, sex, food preferences, wearable or mobile device data, food allergies, health goals, biometric data (such as height, weight, blood pressure measurement, blood test results), genetic and other -omic data, one or more medical risk factors (medical conditions such as high blood pressure or diabetes), and one or more lifestyle risk factors (such as whether the user uses tobacco products and how much physical activity the user typically gets).

According to another preferred embodiment, one or more suggestions for alternate food items that are equal to or more healthful for the user compared to the user's original selections are provided to the user. The suggested food items to aid the user in selecting food items that are better suited to the user and/or to aid the user in meeting a health goal, personal goals, and informed shopping experience.

According to another preferred embodiment, a system and method provides a health assessment and nutrient profile of a user's current and past food selections or searched foods during a shopping event or as part of planning a shopping event, in order to provide the user with suggestions for alternative food selections based on such information to drive the user toward healthier food options and/or to aid the user in meeting a health goal. Health assessments and other criteria of the food selections, also referred to herein as food scores, may be achieved using a food health and attribute scoring system or method, alone or in combination with information retrieved regarding prior purchases. Food scores are objective assessments of general healthfulness and other attributes of a food item and are not related to any specific user criteria. A user's current selections are preferably ranked according to the food score for each item so the user may assess whether the food items should be included in a shopping cart or list. Most preferably, based on the food scores of one or more of a user's current food selections, one or more alternative food items that have an equal or better food score than one or more of the current food selections are suggested to the user. This provides the user with greater variety in food choices and more healthful food choices.

According to yet another preferred embodiment, the food scores of the current food selections are coupled with the user criteria (from personal survey data) and/or one or more objective health criteria or rules, to provide customized and more accurate assessments of the current food selections and alternative food suggestions to the user. In this embodiment, the one or more alternative suggestions will preferably have a food score that is equal to or better than the food score of the current food item and will be compatible with one or more of the user's criteria and/or objective criteria. For example, if the personal survey data indicates the user has hypertension, then the alternative food item suggested may have lower sodium levels than the current food item. As another example, if the personal survey data indicates the user is vegan, then the alternative food item would be a vegan option. According to another preferred embodiment, the user may select one or more of the alternative food items, which will automatically replace the current selection in the user's online cart or shopping list.

A system and method according to another preferred embodiment also provides an overall healthfulness score for items purchased or added to a cart or list by a user, based on the specific foods items selected by the user. Preferably the overall score is based on the food scores for individual food items purchased, but other scoring criteria and other scoring categories may also be used to show the user his or her relative performance in making more healthful selections over time. The overall scoring may also include scoring elements based on the user's personal survey data, such as a health goal or improvements in biometric data. Most preferably, a system and method according to a preferred embodiment tracks the user's progress in improving the user's overall score by comparing a current food purchasing score to one or more previous food purchasing scores and/or averaging a user's food purchasing scores over a period of time. This allows a user's previous food purchases and health assessments of those purchases to be tracked over a period of time for comparison to the current selections and/or to other previous food purchases to show the user whether the user is making better food choices over time. Most preferably, a simple and easily understandable visual scale, such as a graphic meter, is used to provide the user with the tracking data and/or overall food purchasing score. Providing the user with easily understandable evidence of whether his/her food choices are improving over time provides incentive for the user to continue making healthy choices or begin making better choices to improve the overall food purchasing score.

According to another preferred embodiment, a system and method according to the invention also tracks a first user's food selection choices, overall scoring, or both and compares it to one or more other user's food selection choices, overall scoring, or both to provide the first user with an indicator of how his/her choices compare to other user's choices. Most preferably, the first user and the other users in the comparison are grouped by one or more common attributes, such as age range, location (such as city or zip code), or medical condition. This allows the first user to compare his/her choices to the choices of similarly situated users in a pseudo competitive manner to provide incentive for the first user to continue making healthy choices or begin making better choices to improve his/her score relative to other users.

According to another preferred embodiment, a system and method according to the invention allows a user to input personal biometric data (such as a blood pressure reading or weight, a wearable device (such as a FitBit®) or mobile application data (such as an app that tracks steps per day or distance walked)) related to one or more health goals (such as lowering blood pressure or losing weight) and tracks a user's progress in achieving one or more health goals. According to yet another preferred embodiment, the biometric data may be obtained from third party sources via linking of the system according to this embodiment of the invention with the source, such as a doctor's office or medical equipment, a wearable device (such as a FitBit®) or mobile application data (such as an app that tracks steps per day or distance walked). The biometric data may be tracked and the user's personal survey data is preferably automatically updated with new data, which may then be used in making alternative food suggestions to the user.

According to another preferred embodiment, a system and method of the invention provides the user with general health related information, such as educational support materials, corresponding to the user's specific medical risk factors, biometric data, medications, and/or user health goals. According to another preferred embodiment, a system and method of the invention provides the user with generalized health information and tips for improving health generally, which are not related to any user specific medical risk factors, biometric data, medications, and/or user health goals.

According to another preferred embodiment, levels of evidence and nutrient content can be accessed by users as a decision aid. According to another preferred embodiment, users may learn new recipes, healthful tips, suggestions, and techniques in preparation for/during food selection and shopping. Other preferred embodiments include one or more of the following steps or components: (1) User data stored/pulled from survey database (profile information including user demographics, personal medical history, medications, user food survey responses, prior cart purchases (if available), genomics and other -omic data, biometric, microbiome, and activity tracker data) to deliver optimized food recommendations before or during the purchasing experience; (2) Applying personalized food selection nudges based on user goals and needs at defined and variable intervals during the user's food shopping and food consumption intervals to enhance behavior change in food selection, purchasing behavior and consumption of healthier food items; (3) User selects food category (fruits and vegetables) or manual item search (apples) to display options or display past purchases in lists or periodic batches (last month); (4) Last user food item purchased pulled up from database to display and scored; (5) Last user food item attributes compared to food attributes database to match most relevant list of options that meet user needs or preferences; (6) Last food item search rank curates display of food items based on relevancy to user needs and food attributes; (7) Match food purchased to food attribute database to identify nutrition gaps and provide user: (i) food recommendations to close the nutrient gap, (ii) alert to medication interactions, (iii) alert to allergies, (iii) provide personalized nudges to promote purchase of heathier options, and/or (iv) provide educational information to increase awareness; (8) Match prior food item cart purchase (last cart purchase by item) and sort up one level (adjusted rank moves from B to A food, C to B etc.) and foods sorted by user attribute/criteria relevancy; (9) live or pre-recorded coaching based on a user's food searches, previous purchases, habits and/or patterns determined from use of the system and method, searches, previous purchases, and/or biometric data; (10) Food items displayed offer option for “Switch” function to allow user to see options (Display apple and show apple sauce, died apples, apple juice); (11) Food items displayed and offer option for “remix” function to allow user to get the same nutrient mix or range from different foods (remix from Apples to Avocados); (12) Food items displayed and offer option of recipes and add ingredients to cart; (13) utilization of primary food attributes comprising one or more of: (i) sodium, added sugar, calories, vitamins, protein, carbohydrates, total fat, (regulated food label requirements); and (ii) additional food attributes like nitrates, type of fatty acids, and micronutrients essential for optimal health (types of fatty acids, Docosahexaenoic Acid (DHA), zinc, selenium, types of B vitamins, vitamins K & E, sulfur, chloride, magnesium, among others); (14) Utilizing primary food attributes to displayed food scores (such as a letter grade A, B, C, D, E or similar display) for items; (15) utilization of secondary food attributes or preferences for each food item/serving comprising one or more of: taste, texture, engineered food (e.g. Impossible meat), Non-Genetically Modified Organism (GMO), Certified Organic, food additives, artificial ingredients, no preservatives, no pesticides, sustainably harvested, carbon dioxide footprint, ethically produced; (16) utilization of tertiary food attributes or preferences and/or shopping preferences comprising one or more of: non-nutrient attributes like price, sale items, highlighting in-season fresh food items, food freshness verification (meats, fruits and vegetables), food satiety ranking and other non-nutrient attributes relevant to user's food choices and foods shown to support medical conditions in certain combinations of nutrients to deliver optimal combinations associated with health; (17) applying the food selection primary attributes to the secondary and/or tertiary food attributes to curate and display foods that meet user needs and preferences; (18) utilizing icons or graphics that denote one or more of the primary, secondary, or tertiary food attributes/preferences to provide a graphical representation to a user that such attribute/preference is met by a particular food item; (19) connected to grocer food inventory systems to pull available food stock and display food and non-food items; (20) functionality of the in-store enhanced digital experience using a mobile device or internet connected device (for example, allowing a user to take picture of a Universal Product Code (UPC) using a smart phone camera to display a curated selection of foods and food attributes of individual food items, allowing a user to search for food options, obtain information and offers, and/or providing in-store food games to enhance in-store shopping experience for users and their family members); (21) works for dining out and convenience stores to curate options to meet user food consumption needs for optimal health; (22) uses machine learning to curate increasingly personalized and relevant options for users based on past shopping and purchasing behavior to present more relevant food options and personalized nudges to promote better selections; (23) can display “new” store options first in curated food display to users; (24) Interface with other Application Programming Interfaces (API) to allow for importation of other databases and datasets such as: blockchain food tracking “farm to fork”; (25) Food story video display for food producers to promote products direct to users; (26) ‘Food kudos’ to display food attributes directly to users (Non-GMO, Organic); (27) Calculate and issue reward badges and coupons to incentivize healthier food shopping; (28) Curate food meal kits based on user criteria and food attributes; (28) track and produce individual and aggregate food consumption reports based on scores and food purchasing patterns for research purposes; and (29) Deliver food coaching and nutrition support (chat with nutritionist) function.

### BRIEF DESCRIPTION OF THE DRAWINGS

The method of the invention is further described and explained in relation to the following drawings wherein:

FIG. 1 is a flow chart showing process stages for a preferred embodiment of the improving food selections system and method of the invention;

FIGS. 2A-2B are continuing flow charts showing preferred process stages and steps for the embodiment according to FIG. 1;

FIG. 3 is a flow chart showing preferred process sub-steps for a step of the embodiment according to FIGS. 1 and 2A-2B;

FIG. 4 is a flow chart showing preferred process steps for a stage of the embodiment according to FIGS. 1 and 2A-2B;

FIG. 5 is a flow chart showing preferred process steps for a stage of the embodiment according to FIGS. 1 and 2A-2B;

FIGS. 6A-6B are continuing flow charts showing preferred process steps for a stage of the embodiment according to FIGS. 1 and 2A-2B;

FIGS. 7A-7B are continuing flow charts showing alternative preferred process steps the stage of the embodiment according to FIGS. 6A-6B;

FIG. 8 is a flow chart showing preferred process steps for a stage of the embodiment according to FIGS. 1 and 2A-2B;

FIG. 9 is a flow chart showing preferred process steps for a stage of the embodiment according to FIGS. 1 and 2A-2B;

FIG. 10 is a flow chart showing preferred process steps for a stage of the embodiment according to FIG. 1;

FIGS. 11A-11C are continuing flow charts showing preferred process steps and stages for another preferred embodiment of the improving food selections system and method of the invention;

FIGS. 12A-12C are continuing graphical representations of food and shopping related information displayed to a user according to one preferred embodiment of the invention;

FIG. 13 is a graphical representation of rewards information displayed to a user according to one preferred embodiment of the invention; and

FIG. 14 is a graphical representation of user profile information that may be entered by a user according to one preferred embodiment of the invention.

### DESCRIPTION OF THE PREFERRED EMBODIMENTS

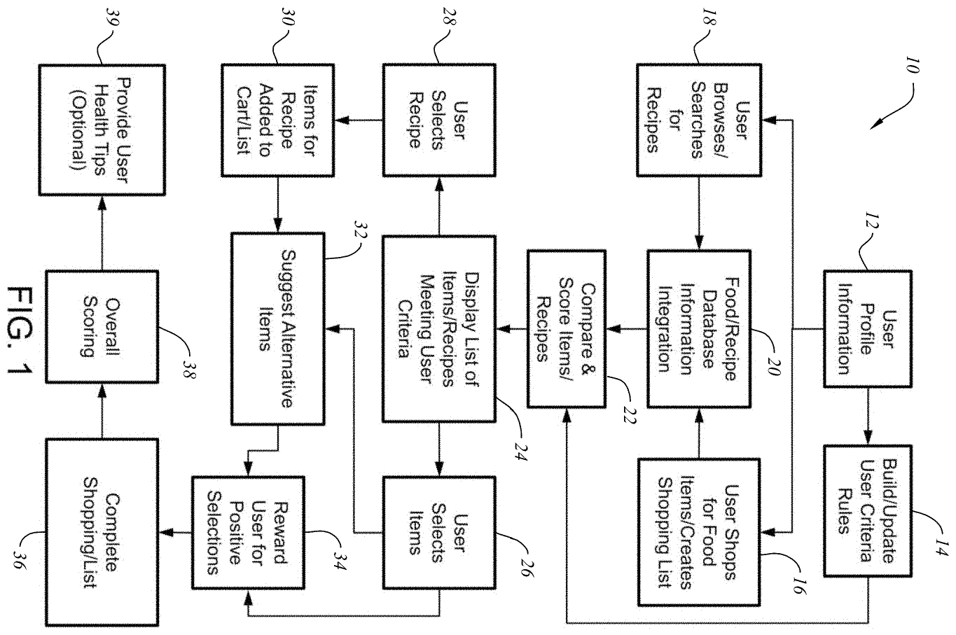

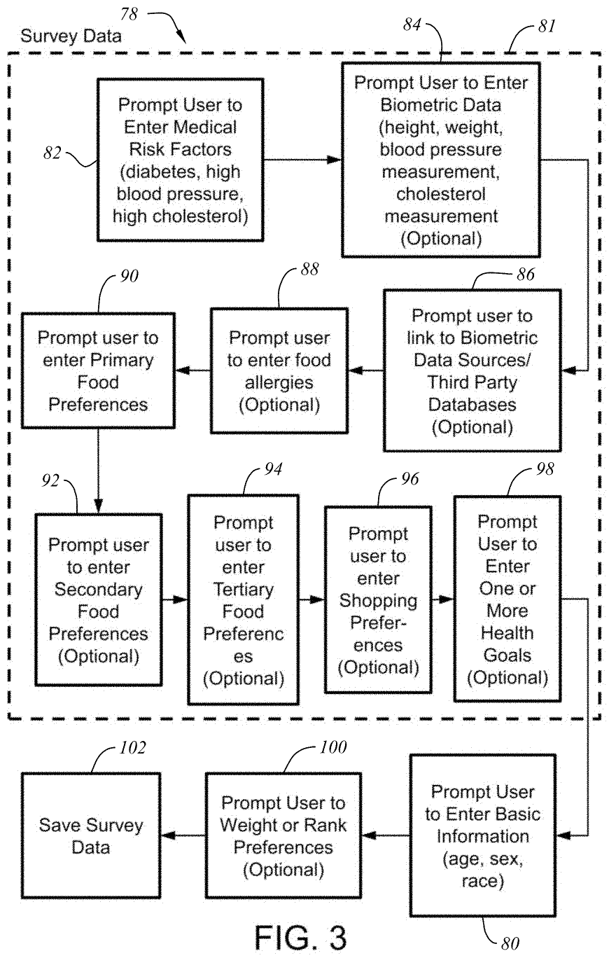

A preferred embodiment of a method or system 10 for improving food selections is shown in FIGS. 1-10. The method or system 10 includes several processing stages, including entry of a user's personal profile information 12, building/updating a user's criteria 14, a user shopping for food items/creating a list of food items 16, a user browsing/searching for recipes 18, integration with one or more food or recipe information databases 20, comparing and scoring items/recipes 22, displaying a list of items/recipes meeting at least some of the user's criteria 24 and preferably ranked in order of a food score for each item, a user selects a recipe 28 from the displayed list and items for the recipe are added to the user's shopping cart or saved list 30, a user selecting items from the displayed list 26, suggesting alternative food items 32 (preferably of the same or a higher food score as the user's originally selected item), rewarding a user for positive selections and/or displaying potential rewards for positive selection 34, shopping/list completion stage 36, optional overall scoring stage 38, and optionally providing health tips stage 39. Method or system 10 may be a stand-alone application for use on a user computing device (such as a computer or smart phone) or may be integrated with an online shopping platform of one or more grocers or other food suppliers, or an ordering platform for one or more restaurants, accessible through a user computing device.

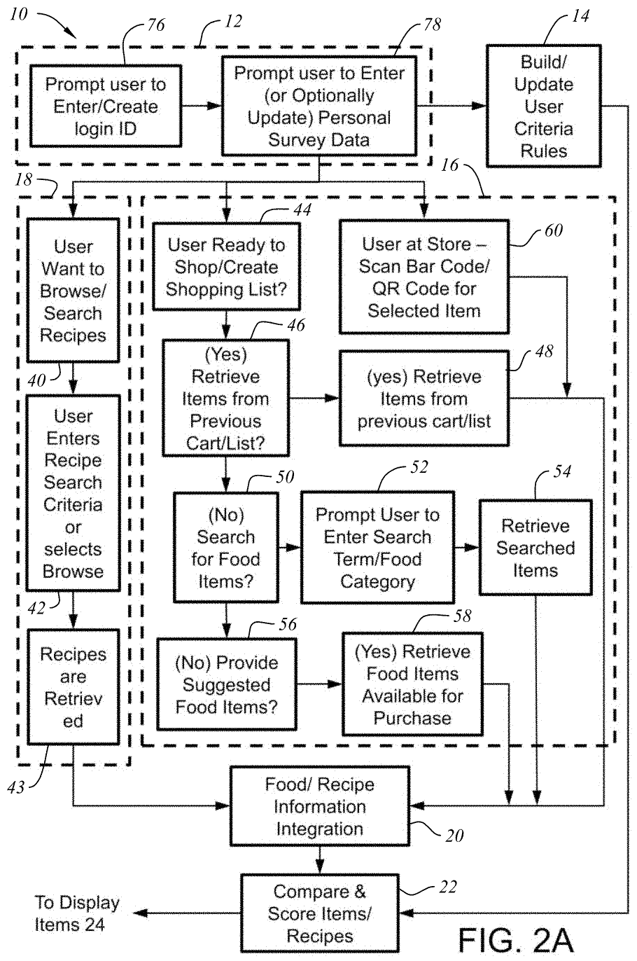

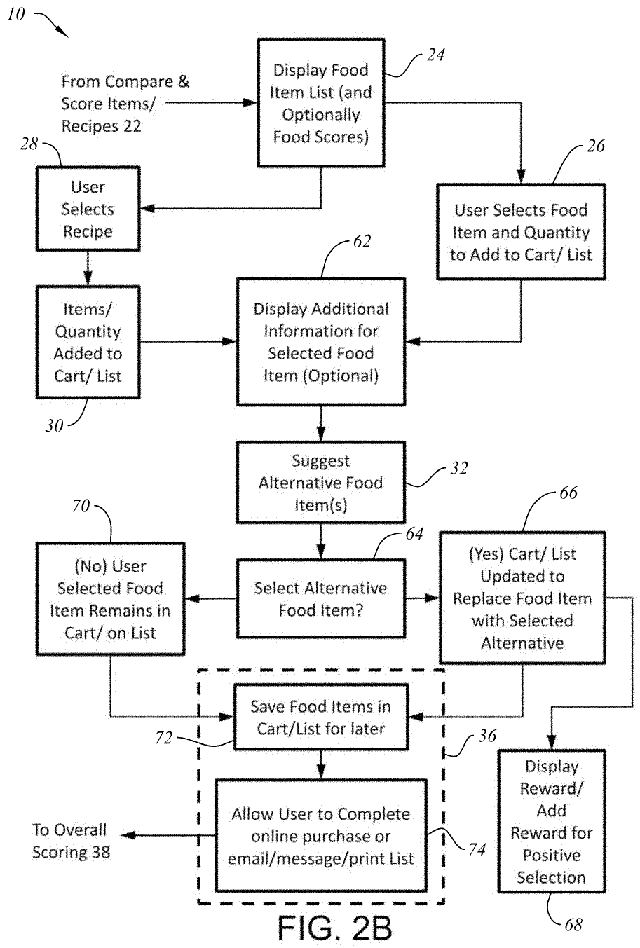

As shown in FIGS. 2A-2B, 3 and 4, a personal profile information entry stage 12 preferably comprises prompting a user to create/enter a login identification (ID) and password 76 and to enter (or optionally update) personal survey data 78. Personal survey data 78 preferably comprises prompting the user to enter basic information 80, such as name, age, sex, race, and optionally address information. Basic address or location information, such as zip code or city and state, may also be obtained through global positioning system (GPS) or other location applications on the user's computing device. A user may also be prompted to enter user criteria 81 comprising one or more of: (1) medical risk factors 82; (2) biometric data 84 (and/or link to biometric data sources 86); (3) food allergies 88; (4) one or more food preferences 90, 92, 94; (5) shopping preferences 96; (6) and/or health goals 98 or optionally to link third-party database connections through an API. This user criteria information 81 is used in comparing and scoring stage 22 to compare the user's criteria to nutrition and other attributes of one or more food items to determine which food items are best suited for the particular user based on a set of user criteria rules 105. Any combination of these user criteria 81 may be used in method or system 10.

Medical risk factors or medical conditions entered at step 82 preferably comprise one or more medical conditions the user has (high blood pressure, diabetes, high cholesterol, for example). A user may also optionally be prompted to enter any prescription or over-the-counter medications that are regularly used, as there may be interferences or incompatibilities with certain food ingredients or foods and these medications. A user is also optionally prompted to enter biometric data at step 84. Biometric data may include the user's historic, recent or average blood pressure measurement, cholesterol levels, height, weight, body mass index (BMI), blood test results, or other biometric data. A user may optionally be prompted to link to biometric data sources and/or other third-party databases at step 86, so that biometric and other data may be communicated directly to into system 10. Biometric data sources may include personal blood pressure or blood glucose monitoring devices that are capable of communicating blood pressure or glucose reading data via a wired or wireless connection, a database for a physician of the user, or a personal health tracker (such as a FitBit®). Other databases that may be linked to system 10 include a pharmacy database to provide information on prescription medications or telemedicine databases. Shopping related databases may also be linked to system 10 through a prompt at step 86 or another prompt in survey data 78 (not shown), so that the user may utilize coupon databases, or shopping rewards databases (such as platforms that provide third party rewards for shopping at particular merchants, with a particular credit card, and/or through a particular website). A user may optionally, but preferably, be prompted to enter any food allergies or sensitivities or both at step 88. The data entered at steps 82, 84, and 88, and any data retrieved through step 86 may be combined together as a user's health factors and general health factors are the types of data that may be entered or retrieved at these steps.

A user is also preferably prompted to enter one or more food preferences at step 90, and optionally 92 and 94. Primary food preferences 90 include one or more nutritional related food preferences to include or exclude, such as: low fat, reduced sugar, gluten-free, no trans-fats, particular vitamin or mineral content, etc. One or more primary food preferences 90 may overlap with one or more food sensitivities at step 88, for example gluten sensitivity and gluten-free food preference. Secondary food preferences 92 include one or more preferences related to the type of food to include or exclude, such as vegetarian, vegan, no red meat, no seafood, no dairy, etc. Tertiary preferences 94 include one or more preferences related to food production to include or exclude, such as in-season fruits and vegetables, certified organic, non-GMO, locally sourced, free-range meat, pesticide use, no high fructose corn syrup, preservative free, etc. Preferences 90, 92, and 94 may be combined into a single category or separated into one or more preference categories, as desired. If a user typically shops for friends or family members in addition to the user, the user may include medical risk factors, biometric data, medications, and food preferences of such friends or family member in the user's own profile or can create separate profiles for pets, friends, family members, or those within their household that they normally shop for. The user can select which of the user, friend(s), and/or family member(s) system 10 should take into account when building/updating user criteria rules 14 for a particular shopping trip or list.

A user is also optionally prompted at step 96 to enter shopping preferences, which may include: a preferred grocery store or rank order of local grocery stores, preference for shopping for on sale items, coupon use (or displaying items with online coupons available first), displaying new items first, etc. Shopping preferences 96 may be integrated with food preferences 90, 92, 94, or may be separate. A user may enter one or more health goals or health plans at step 98, such as: weight loss, increasing fruit and vegetable consumption, gluten free diets, low blood pressure diets, published scientifically backed diets, reducing red meat consumption, reducing sodium consumption, reducing sugar consumption, etc. A health goal 98 may also include a desire to reduce consumption of one or more particular ingredients (e.g. sodium, saturated fats, etc.) or types of foods (e.g. red meat or dairy), to increase consumption of one or more particular ingredients (e.g. protein) or types of foods (e.g. fruits or vegetables), to lose weight or reduce calorie consumption, change biometric data (e.g. lower blood pressure), etc. and any reduction or increase may be based on a given number of servings of such ingredients or types of food over a given time period. A user may also be prompted to enter basic information (such as name, address, race, sex, age) at step 80. This step may be carried out before step 82, but may be carried out at any time. A user may also optionally provide weighting or ranking information at step 100 to indicate the relative subjective importance to the user of one or more of the user criteria 81 compared to other criteria 81. For example, a primary food preference 90 may be more important to the user than a secondary food preference 92 and a shopping preference 96 may also be more important to the user than a secondary food preference 92. A weighting or ranking of importance at step 100 will later be used to create subjective weighting or ranking rules at step 116 to be used in comparing food items in stage 22. In some scenarios, a preferred primary food preference 90 may include a dietary plan of which the user plans to adhere to which will affect ranking of importance 100 and rules 116 when displayed to the user 24. A user's survey data is saved at step 102. Each time a user logs into system 10, or at periodic login intervals (such as every month or every three months, for example), the user may optionally be prompted to update survey data 78, and particularly user criteria 81, which allows the user to change any survey data 78 that has changed since the last time data was entered. Alternatively, a user may manually select to update survey data 78 by selecting such on a user display interface on the user's computing device. If system 10 is linked to biometric databases or devices at step 86, system 10 preferably periodically retrieves new data from such databases or devices at pre-set or user selected intervals and automatically updates the user's survey data. Alternatively, a user may manually select when to have system 10 and a biometric device communicate with each other.

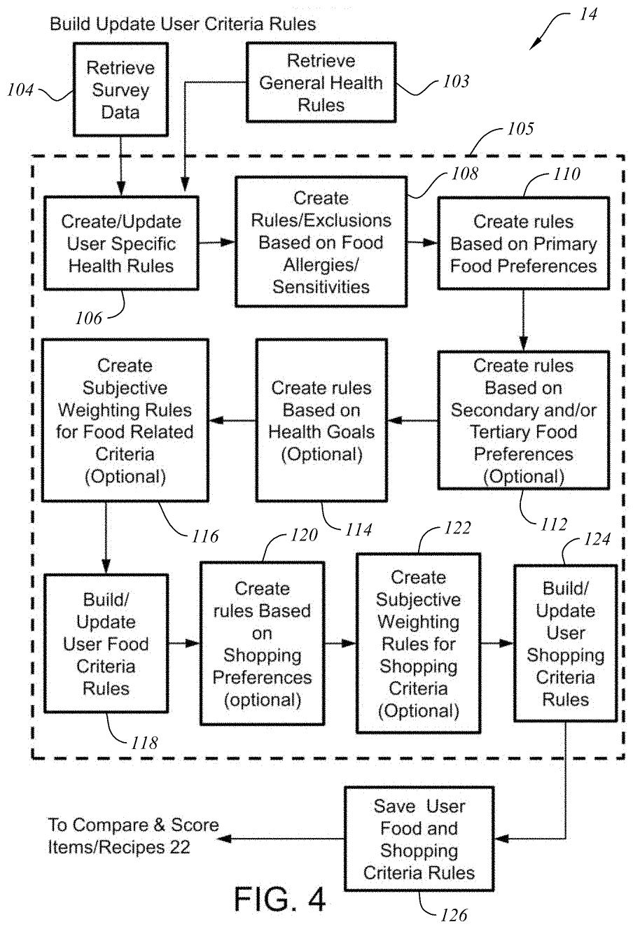

As shown in FIGS. 2A-2B and 4, a build/update user criteria rules stage 14 preferably comprises retrieving a user's survey data 104 and creating (or updating if there has been a change in user's survey data 78 since the last set of user criteria rules 14 were created) one or more rules to be applied to food items based on user criteria 81. A rule is used to exclude or include a particular food item, or in combination with subjective weighting/ranking at step 116 and/or objective weighting/ranking at step 156 to raise or lower a rank of a particular food item, in a list 24 to be displayed for the user. One or more user criteria rules comprise: (1) user specific health rules 106; (2) rules based on food allergies and/or sensitivities 108; (3) rules based on one or more food preferences 110, 112; (4) rules based on health goals 114; and/or (5) rules based on shopping preferences 120.

System 10 preferably comprises one or more general health rules that apply to various medical risk factors, medical conditions, or medications that may be taken. These rules are generalized and not user specific. For example, a general hypertension rule may include food items with sodium levels per serving that are less than or equal to a predetermined first threshold and/or exclude food items with sodium levels above that first threshold. One or more general sub-rules may also be used. For example, for a blood pressure reading above a predetermined threshold (such as one that may be borderline high), a sub-rule may include food items with sodium levels per serving that are less than or equal to a predetermined second threshold and/or exclude food items with sodium levels above that second threshold, where the second threshold is the same as the first threshold or different than the first threshold (in this particular example, the second threshold may be higher than the first threshold). Similar general health rules may be created for various food ingredients that may impact various other medical risk factors or conditions, such as sugar thresholds for users with diabetes, cholesterol thresholds for users with high cholesterol, fat thresholds for users with hyperlipidemia, etc. or that may interfere with or be incompatible with certain medications, such as medications that should not be taken with grapefruit or grapefruit juice, for example. These general health rules are retrieved at step 103 and are used, along with the retrieved user survey data from step 104, to create/update user specific health rules 106, each of which factor into ranking 100, rules 116, and items displayed for purchase to the user 24.

Most preferably, user specific health rules 106 are based on user's medical risk factors/medical conditions 82, biometric data 84, 86, and/or medications being taken and various food ingredients that may impact those conditions or medications from the retrieved general health rules 103. The rules and sub-rules retrieved at step 103 are generalized in that they apply to all users having the same or similar medical conditions or biometric data or medications; however, the user specific health rules 106 are a user specific collection of one or more generalized rules or sub-rules 103 based on the user's particular health or medical conditions and biometric data. For example, if a user has hypertension, then user specific health rules 106 will include the general health rule related to hypertension (e.g. to include food items with sodium levels per serving that are less than or equal to a predetermined first threshold and/or to exclude food items with sodium levels above that first threshold). If user's survey data 78 does not list high blood pressure/hypertension as a medical risk factor but the user's biometric data indicates the user's blood pressure is borderline or near a high range, then a sub-rule for the range of blood pressure readings within which the user's reading falls would be included in the user specific health rules 106 (e.g. to include food items with sodium levels per serving that are less than or equal to a predetermined second threshold and/or to exclude food items with sodium levels above that second threshold, where the second threshold is the same as the first threshold or different than the first threshold). As another example, if a user blood test results indicate the user is deficient in a particular vitamin, mineral or other nutrient, then a rule 106 may be designed to include food items with higher levels of such vitamins, minerals, or other nutrients or to rank them higher in a weighted list of food items for the user or suggested for the user. Similar user specific health rules 106 rules may be created based on the user's medical risk factors 82 and/or biometric data 84 and/or 86. Food preferences 90, 92, 94 may also be applied with specific health rules 106, and generalized health rules 103 to allow for personalized rank ordering of food products displayed to the user 24. In the event of preferences entered at 90, 92, 94 being out of range of both specific health rules 106 and generalized health rules 103, certain out of range products may or may not be displayed back to the user 24 with a notification that a product is outside of their health goals or dietary adherence plan.

If the user indicated any food allergies or sensitivities at step 88, then one or more rules are preferably created (or updated) at step 108 based on those allergies or sensitivities. Most preferably, the rule will exclude any foods that contain an allergen for the user from being displayed to the user at step 24, to avoid any negative health impacts on the user. Alternatively, a rule may allow a food item containing an allergen to be displayed at step 24 along with a warning label and/or displayed at or near a bottom of a list of foods (assigned a lower rank at step 148 based on the allergen and limit 108). Similarly, a rule 108 may exclude any foods that contain an ingredient to which the user has a sensitivity from being displayed to the user at step 24 or may allow such item to be displayed with a warning label and/or displayed near a bottom of a list of goods (assigned a lower rank at step 148 based on the food sensitivity rule at 108).

Food preference rules are preferably created (or updated) at steps 110 and optionally 112. Food preference rules 110 are based on user's primary food preferences 90 and optional food preference limits 112 are based on user's secondary and/or tertiary food preferences 92, 94, to exclude or include food items based on the preferences returned back to the user on search M. Rules in steps 110 and 112 may be combined into one step or one set of rules based on all the food preferences but, are most preferably broken down into two or more categories based on the type of food preference (primary, secondary, tertiary). As an example of a primary food preference limit 110, if a user indicated a primary food preference 90 for lower fat foods, then a rule may be applied to include food items with saturated or total fat levels per serving that are less than or equal to a predetermined first threshold and/or to exclude food items with saturated or total fat levels above that first threshold. As an example of a secondary food preference limit 112, if a user indicated a secondary food preference 92 of vegan, then food items that are not vegan may be excluded from the list.

Health goal rules are preferably created/updated at step 114 based on user's health goals 98. For example, if the user has a health goal of weight loss, then a health goal limit may include foods with total sugars per serving that are less than or equal to a predetermined threshold, or food items with without added sugars, or food items labeled as “reduced sugar. Similar rules may be set based on total calories per serving and/or fat per serving, for example. Additionally, other rules may be set to increase or decrease consumption of particular ingredients or types of foods based on a user's health goals and may be combined with other rules and food preferences.

To ultimately provide a greater number of food items for the user to be displayed for the user at step 24, the sub-rules from steps 110, 112, and 114 are preferably subjectively ranked or weighted at step 116 based on subjective weighting/ranking criteria selected by the user at step 100 to arrive at a user's food criteria at step 118. Subjective weighting/ranking at step 116 is optional, but preferred. As an example, if a user indicated at step 100 that having certified organic foods is more important than locally sourced foods, then at step 116 that preference is built into the user food criteria 118 so that at step 144 preference is given to organic items over locally sourced foods. For example, if item A is organic and local, item B is non-organic and non-local, item C is non-organic and local, and item D is organic and non-local, then the rules applied in step 144 according to the user's preference for organic over local items as set in step 100 would rank these items in the order of the user's preference as item A, D, C, and then B. Alternatively, items C and B may be excluded from the list or flagged as not meeting a criteria(s), as further discussed below. When multiple food criteria are weighted or ranked at step 100, then numerical values are preferably assigned to the criteria to build the user food criteria at step 118, as will be understood by those of ordinary skill in the art.

Although the rules in steps 106 and 108 may also be weighted or ranked, they are most preferably not weighted or ranked by a user at step 100 (but may be ranked or weighted according to pre-determined rules at step 150), and are treated as hard requirements. If they are weighted or ranked, they are most preferably weighted or ranked based on an algorithm that factors in medical criteria at step 150 and not based on user selections at step 100.

System 10 may be configured to only apply a limited number of the rules from steps 110, 112, and 114 (and optionally from steps 106 and 108), with those rules that are ranked the highest in steps 116 (based on user selected criteria at step 100) and/or 148 (based on objective criteria) taking preference in the order of operation at steps 118 and/or in applying/comparing the rules to particular food items at step 144 and/or 148. Various combinations of rules may be applied to medical conditions, food preferences, and health goals, and different weighting criteria may be used, to arrive at user food criteria at step 118 as will be understood by those of ordinary skill in the art.

Rules based on shopping preferences are created/updated at step 120. For example, if a user indicates a preference for viewing new items first, then a rule based on when an item was first introduced or added to a food database (such as the database of foods available for purchase from a particular grocer) will be created so that newer items show up higher in a display list at step 24 of food items meeting other rules. Other shopping preference rules may include display food items that are on sale and meet other rules higher than items that are not on sale. To ultimately provide a greater number of food items for the user to be displayed for the user at step 24, the sub-rules from step 120 are preferably ranked or weighted at step 122 based on user's selected weighting/ranking criteria from step 100 to arrive at a user's shopping criteria at step 124. Weighting/ranking at step 122 is optional, but preferred. As an example, if a user indicated at step 110 that having on sale food items is more important than new items, then at step 124 the rule related to displaying on sale items will be carried out before the rule for new items. System 10 may be configured to only apply a limited number of the rules from step 120, with those rules that are ranked the highest at step 100 taking preference in the order of operation at step 122.

When a user is logged into system 10, the user has the option of browsing or searching for recipes at step 18 or shopping/creating a list at step 16, as shown in FIGS. 1-2A. These options may be provided for selection by the user or system 10 may prompt user as to whether the user wants to search for recipes at step 18, and if the user enters (1) “yes,” system 10 will proceed to step 42 or (2) “no,” system 10 will proceed to step 16 (step 44 or step 60). These prompts may be presented in any order. Step 16 preferably comprises a user shopping or creating a shopping list online at step 44 or a user scanning items at a store at step 60. Most preferably, system 10 is integrated with an online shopping platform for a particular grocer or store, that allows a user to: add items to a cart or shopping list, complete a purchase for later pickup or delivery, save a list for printing or later purchase, or later electronic use during an in-store shopping trip, apply coupons, search for food items, search for recipes, browse food items, browse recipes, review sale items, and saves a user's shopping history (such as previous cart purchases or previous shopping lists), but system 10 may also be a stand-alone system. If it is a stand-alone system, system 10 would not allow a user to complete an online purchase, but could still allow a user to create lists for shopping at a particular grocer or store in-person or online and to retrieve information about various items on the user's list or other items that the user selects while in-store; or alternatively refer the user to either the grocer's online website for order completion or paying for cart items through a third-party payment processor. For example, if a user is at a store, a bar code or QR code for a particular item the user is interested in purchasing may be scanned at step 60. Nutritional, use in recipes, switch, scoring, and other information regarding that item is retrieved through food information integration stage 20, discussed further below.

If a user is shopping or creating a list online, the user is preferably prompted to indicate if the user would like to retrieve items from a previous list or shopping cart (if there is a previous list or shopping cart) at step 46. If yes, then the user's previous shopping cart items and/or shopping list are retrieved at step 48 and can either be re-purchased or provide nutritional and other information regarding the items from the previous cart/list which are retrieved through food information integration stage 20, discussed further below. If the user does not have a previous list or shopping cart (such as for the first time the user is using system 10 or using system 10 in conjunction with a particular store's online shopping platform), or the user indicates he/she does not want to retrieve items from a previous list or shopping cart, then the user is prompted to search for food items at step 50. A user is preferably prompted to enter a food category (e.g. dairy, pasta, meat, vegetables, etc.) or to enter a particular food item or brand name (e.g. spaghetti, apples, Dannon® yogurt, etc.) at step 52. The searched items are retrieved at step 54 and nutritional and other information regarding those items is retrieved through food information integration stage 20, discussed further below. A user may also request that food items be suggested for the user at step 56. Items that are available for purchase through a particular grocer's online shopping platform are retrieved at step 58, and preferably one or more predetermined rules according to health criteria (most preferably general health criteria that are not user specific) are applied to reduce the number of items retrieved at step 58. Nutritional and other information regarding the retrieved items is retrieved through food information integration stage 20, discussed further below. If a user wants to browse or search for recipes at step 40, the user is prompted to enter search criteria (e.g. main courses, sides, recipes using beef, vegetarian recipes, etc.) or to request suggested recipes at step 42. For suggested recipes, preferably one or more predetermined rules according to health criteria (most preferably general health criteria that is not user specific) are applied to reduce the number of recipes retrieved at step 42. Nutritional and other information regarding the retrieved items is retrieved through food information integration stage 20, discussed further below.

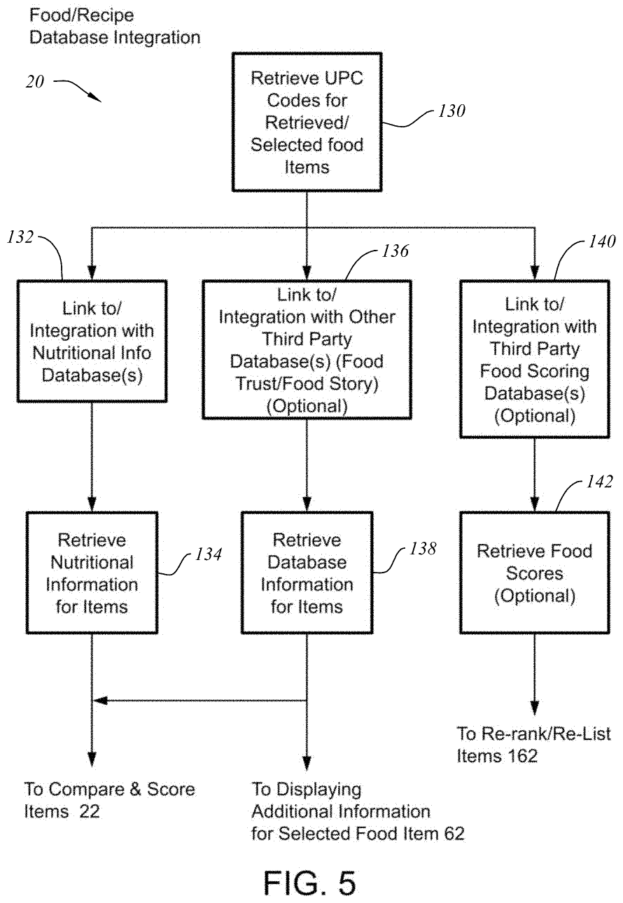

As shown in FIG. 5, food/recipe database information integration stage 20 preferably comprises retrieving a UPC (or other identifying information) at step 130 for items that are retrieved in steps 48, 54, or 58 and/or for ingredients included in a recipe retrieved at step 42. For an item selected and scanned while at a store at step 60, the UPC bar code is typically what is scanned, but if a QR code is scanned, it would then be used to retrieve the UPC for the item at step 130. The UPC is then used to link to a database containing nutritional information regarding the item associated with the UPC at step 132 and to retrieve primary food attributes of that item at step 134 (similar to step 400 in system 310 described below). Most preferably, this database includes primary food attributes related to nutritional information, which preferably include per serving information regarding calories, protein, carbohydrates, fats (per type), fiber, added sugars, vitamins, minerals, nitrates, preservatives, and/or additives. This database also includes product pricing details and coupon or other discount programs managed by a particular grocer or consumer product manufacturer. This database may be a database integrated into system 10 or system 10 may be configured to retrieve this information through integration with a third-party database, such as Label Insight.

Food/recipe database information integration stage 20 also optionally, but preferably, comprises linking to or integration with one or more other databases containing information regarding one or more other attributes of the food items at step 136 and retrieving information regarding a secondary, tertiary, and/or higher level attributes for the food items at step 138. For example, a secondary food attribute (similar to step 402 in system 310) may include information regarding micronutrients (e.g. choline, biotin, lutein), food-drug interactions, and nutrients associated with managing health conditions, levels of scientific evidence may also be provided in this stage. A tertiary food attribute (similar to step 404 in system 310) may include information regarding food production and manufacturing that may have health implications, such as whether the retrieved/selected food item is non-GMO, certified organic, produced with or without the use of pesticides, etc. One or more other higher level food attributes and/or other information regarding the food item or recipe (similar to steps 406, 408 in system 310) may also be retrieved at step 138, such as information regarding the geographic location where the food was produced, when it was harvested or produced, how it was shipped to the store, whether it is an in-season produce item, whether it has ever been frozen, whether it is the subject of a current or past recall, etc. Other non-food related attributes of a food item, such as whether the item is a new item or is on sale may also be retrieved at step 138. With respect to recipes, as an alternative to retrieving food attribute information for the ingredients in a retrieved recipe, steps 134 and/or 138 may be used to retrieve pre-determined/pre-calculated information regarding the recipe, such as totals of primary food attributes per serving of the recipe. The information retrieved at steps 134 and 138 is then used in comparing and scoring items/recipes stage 22 or 22A. The information retrieved at step 138 may also optionally be displayed to a user at step 62 (FIG. 2B).

Food/recipe database information integration stage 20 also optionally comprises linking to or integration with a third-party food scoring database at step 140. Such a database may include a score for the particular food item retrieved/selected. The score may be in any format, but is preferably easy for the user to understand, such as a letter grade, color grade, a scale of stars or other icons, or a numerical grade or any combination of the previous listed. Once linked to a scoring database at step 140, the food scores are retrieved and step 142 and used in stage 22. Food scores from database 140 retrieved at step 142 are most preferably general food scores based on the overall health associated attributes of a food generally, and not related to any user specific health criteria or preferences, which are preferably separately taken into account in comparing and scoring stage 22.

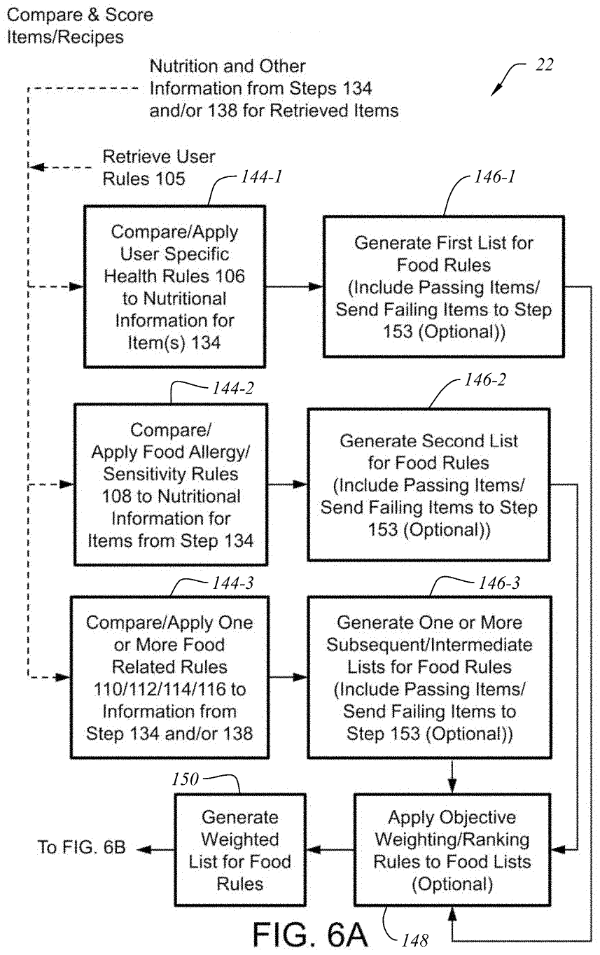

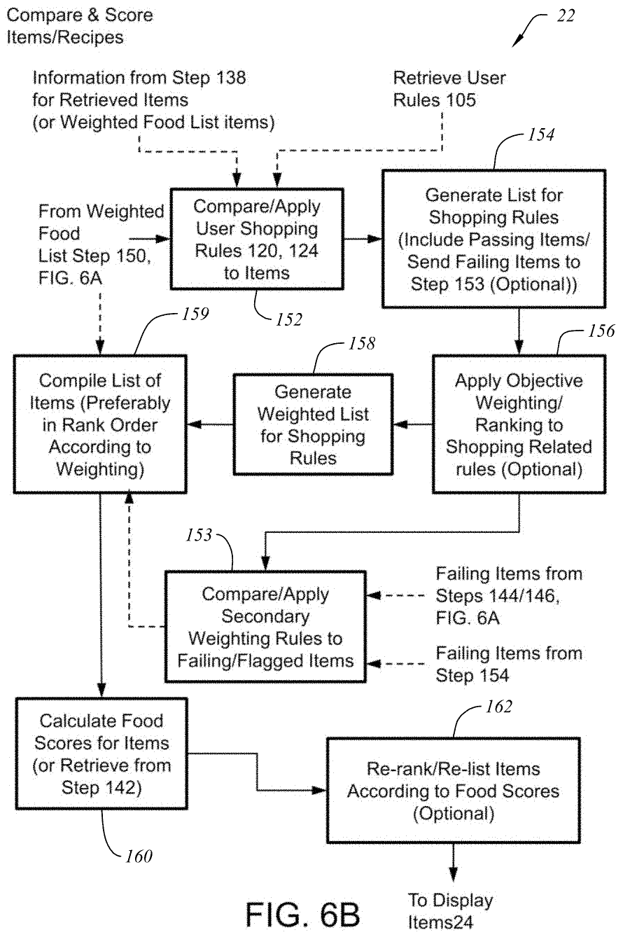

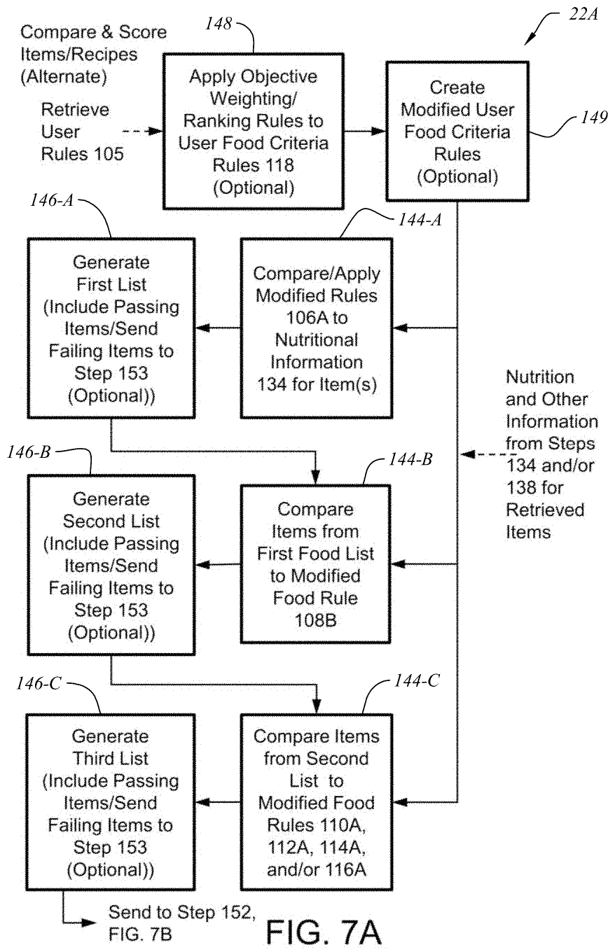

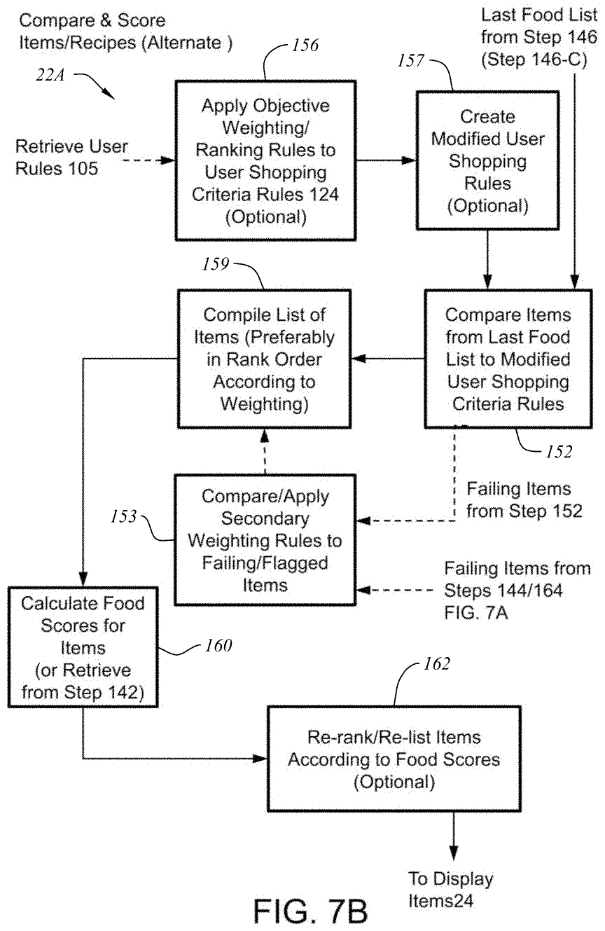

As shown in FIGS. 6A-7B, comparing and scoring stage 22 or 22A (alternate) preferably comprises steps that compare food items or recipes retrieved from steps 48, 54, 58, 42 or selected at step 60 (or alternative food items retrieved in stage 32 discussed below) to one or more of the user's criteria 81 using the rules created in stage 14 (a user's food criteria rules from step 118 and the user's shopping criteria rules from step 124) and/or objective weighting rules 148, 156. A list of food items that best match overall general health criteria (using objective weighting rules 148, 156 and/or food scores 160, 142) and/or user-specific health criteria and preferences (user criteria 81 and/or user weighting/ranking criteria 100) is compiled based on the comparison or application of the rules 118 (or any sub-rule thereof), 124 (or any sub-rule thereof), 148, and/or 156 to various attributes of the food obtained in food/recipe database integration stage 20. Subjective weighting rules 116 and 122 are optional and need not be applied. The food items are preferably ranked according to (1) a match score, indicating how well each item meets the applied rules 118 (or any sub-rule thereof), 124 (or any sub-rule thereof), 148, and/or 156 (such as a percentage of rules that are matched) and/or (2) based on a food score 142, 160. A list 24 displayed to the user preferably shows the food items in order from highest ranking or highest food score (or a combination thereof) to lowest ranking or lowest food score (or a combination thereof).

As shown in FIGS. 6A-6B, stage 22 preferably comprises (1) retrieving user rules from step 105; (2) retrieving information regarding the selected food items from steps 134 and/or 138; (3) comparing or applying user food/health related rules to the information for each of the food items at step 144 (steps 144-1, 144-2, 144-3, etc.); (4) generating a list of food items matching, passing, or meeting the rules as applied in step 144 at step 146 (lists 146-1, 146-2, 146-3, etc.); (5) applying objective food/health related weighting or ranking rules to each of the lists from step 146 at step 148; (6) generating a weighted list of items based on food/health weighting rules from step 148 at step 150; (7) comparing or applying user shopping related rules to the information for each of the food items in the weighted food list from step 150 at step 152; (8) generating a list of food items matching, passing, or meeting the rules as applied in step 152 at step 154; (9) applying objective shopping related weighting or ranking rules to the list from step 154 at step 156; (10) generating a weighted list of food items based on shopping rules from step 156 at step 158; (11) compiling a full list of items from the weighted list of step 158 at step 159; (12) calculating food scores for the items on the list from step 159 at step 160 (or retrieving food scores for those items from step 142); and (13) optionally re-ranking or re-ordering the list from step 159 according to the food scores (higher food scores appearing before lower scoring foods at display step 24) at step 162. The order of comparing the food items to one rule relative to another rule and generating the lists is not critical in stage 22 and the steps may be carried out in a different order. Step (7) may also compare or apply user shopping related rules to the information for each of the food items (rather than the narrowed, weighted food list 150) at step 152. In that case, the results from the weighted list based on food rules 150 and weighted list based on shopping rules 158 are preferably compiled into a single list at step 159. One or more objective weighting rules from steps 148 and/or 156 may be used to resolve any conflicts in the two weighted lists to arrive at a full list 159.

As shown in FIGS. 6A-6B, stage 22 preferably comprises generating successive lists of food items from the retrieved or selected items that pass, match, or meet the rules that are applied. In other words, the comparison steps are applied in parallel, rather than in series and each list generated at step 146 may include duplicate items that are narrowed down and possibly eliminated at weighting step 148. Most preferably, food attributes from steps 134 and/or 138 for all retrieved items (retrieved from steps 43, 48, 54, 58, 60, and/or alternative suggestion items from stage 32) are compared to each of the rules 106, 108, 110, 112, 114, and/or 116 that are applied in system 10. Although not all rules need to be applied in system 10, whichever rules are applied or compared in stage 22 are preferably compared to a complete list of retrieved items, rather than creating successively narrower lists as described below in alternative embodiment of stage 22A. A first list of food items is generated at step 146-1 and includes only items retrieved or selected at steps 43, 48, 54, 58, 60 or from stage 32 that pass, meet, or match all of the user specific health rules 106 at step 144-1. Items that do not pass, meet, or match all of the user specific health rules 106 may be excluded from the list 146-1, but may optionally be sent to step 153 for further consideration. A second list 146-2 includes only items that pass, meet, or match all of the user food allergy/sensitivity rules 108 at step 144-2. Most preferably items that violate or fail a food allergy rule are excluded and not sent to step 153 for further consideration; however, items that violate or fail a food sensitivity rule may be sent to step 153 with a notification of which rule a food product violates, depending on the weighted rank of the rule and preference of the user in user settings. A third list 146-3 includes only items that pass, meet, or match all of the applied other food rules, such as rules 110, 112, 114, and/or the subjective weighting rules 116 at step 144-3; however, each set of rules may be considered in separate comparison steps (e.g. 144-4, 144-5, etc.) to generate separate lists (146-4, 146-5, etc.). Items that do not pass, meet, or match all of the applied rules at steps 144-3 (or 144-4, etc.) may be excluded from the lists 146-3 (or 146-4, etc.), but may optionally be sent to step 153 for further consideration. Typically, there are only a few shopping rules, which can be compared/applied together in a single step 152 to generate a single list 154, but these may also be broken down into sub-steps (e.g. 152-1, 152-2, 154-1, 154-2, etc.).

As shown in FIGS. 7A-7B, alternate stage 22A preferably comprises (1) retrieving user rules from step 105; (2) applying objective food/health related weighting or ranking rules at step 148 to each of the food related rules from step 105; (3) creating a modified set of food related rules at step 149; (4) retrieving information regarding the selected food items from steps 134 and/or 138; (5) comparing or applying modified user specific health rules 106A to the information for each of the food items at step 144-1; (6) generating a first list of food items matching, passing, or meeting the rules as applied in step 144-A at step 146-A; (7) comparing or applying modified user food allergy/sensitivity rules 108A to the information for each of the food items on list 146-A at step 144-B; (8) generating a second list of food items matching, passing, or meeting the rules as applied in step 144-B at step 146-B; (9) comparing or applying one or more other modified user food rules 110A, 112A, 114A, and/or 116A to the information for each of the food items on list 146-B at step 144-C; (10) generating a third list of food items matching, passing, or meeting the rules as applied in step 144-C at step 146-C; (11) applying objective shopping related weighting or ranking rules at step 156 to each of the shopping related rules from step 105; (12) creating a modified set of shopping related rules at step 157; (13) comparing or applying the modified shopping rules to the last list generated from step 146 (e.g. 146-C) at step 152; (14) compiling a full list of items meeting, passing or matching the modified shopping rules at step 159; (15) calculating food scores for the items on the list from step 159 at step 160 (or retrieving food scores for those items from step 142); and (16) optionally re-ranking or re-ordering the list from step 159 according to the food scores (higher food scores appearing before lower scoring foods at display step 24) at step 162. The order of comparing the food items to one rule relative to another rule and generating the lists is not critical in stage 22A and the steps may be carried out in a different order.

As shown in FIGS. 7A-7B, stage 22A preferably comprises generating successively narrow lists of food items based on which items from the originally retrieved list of items meet, match, or pass the applied rule(s) at each comparison stage 144-A, 144-B, 144-C, etc. In other words, the comparison steps are applied in series, rather than in parallel. Most preferably, food attributes from steps 134 and/or 138 for all retrieved items (items retrieved from 43, 48, 54, 58, 60, and/or alternative suggestion items from stage 32) are compared to only one rule/set of rules (e.g. modified rule 106A), and only those items that meet, match, or pass that rule are compared to the next rule (e.g. modified rule 108A). Not all rules need to be applied in system 10, or processing stage 22A, but whichever rules are applied are compared only to items that pass the previous rule (if any) so that successively narrower lists are obtained at steps 146-A, 146-B, 146-C, etc. A first list of food items is generated at step 146-A and includes only items retrieved or selected at steps 43, 48, 54, 58, 60 or from stage 32 that pass, meet, or match all of the modified user specific health rules 106A at step 144-A. Items that do not pass, meet, or match all of the user specific health rules 106A are excluded from the list 146-A, but may optionally be sent to step 153 for further consideration. A second list 146-B includes only items that pass, meet, or match all of the modified user food allergy/sensitivity rules 108A (and that previous passed modified rule 106A at step 144-A) at step 144-B, because step 144-B uses the narrowed list of items from list 146-A for comparison. Most preferably items that violate or fail a food allergy rule are excluded and not sent to step 153 for further consideration; however, items that violate or fail a food sensitivity rule may be sent to step 153. A third list 146-C includes only items that pass, meet, or match all of the applied other modified food rules, such as rules 110A, 112A, 114A, and/or the subjective weighting rules 116A at step 144-C (and that previously passed modified rule 106A at step 144-A and modified rule 108A at step 144-B). Each set of rules 110A, 112A, 114A, and/or 116A may be considered in separate comparison steps (e.g. 144-D, 144-E, etc.) to generate separate, successively narrower lists (146-D, 146-E, etc.). Items that do not pass, meet, or match all of the applied rules at steps 144-C (or 144-D, etc.) are excluded from the lists 146-C (or 146-D, etc.), but may optionally be sent to step 153 for further consideration. Typically, there are only a few shopping rules, which can be compared/applied together in a single step 152 to last food related list (e.g. list 146-C) using modified shopping rules 157 to compile a single list 159, but these may also be broken down into sub-steps (e.g. 156-A, 156-B, 155-C, 155-D, etc.) if desired.

The objective weighting rules at steps 148 and 156 preferably include one or more predetermined rules that assess the importance of one or more particular food related rules or shopping rules in relation to other food related rules and shopping rules, similar to user preference weighting in steps 116 and 122, except that the rules applied in steps 148 and 156 are objective rules, whereas those in steps 116 and 122 are subjective based on the user's ranking criteria in step 100. The objective weighting of food rules at step 148 and shopping rules at step 156 may be combined into a single objective weighting step or may be carried out in separate steps. These objective rules may overlap with one or more of the subjective weighting rules from steps 116 or 122, particularly for shopping preferences, or they may be different. These rules, particularly objective food related weighting rules at step 148 may supersede or negate one or more user subjective weighting rules from step 116. Objective weighting at steps 148 and/or 156 may be as simple as an overall match score, such as a match percentage for the number of rules an item matches or for which the item meets the criteria. For example, if there are 8 food related rules and 2 shopping related rules and the item meets 7 of the rules, then its match rate would be 70% and it would rank above an item that only matched 6 rules. A user's subjective weighting at steps 116 and 122 may be carried out in the same way. Alternatively, and preferably, objective weighting at steps 148 and 156 gives preference to food rules over shopping rules. For example, if there are 8 food related rules and 2 shopping related rules and an item meets 4 food rules and 2 shopping rules, it would rank lower than another item that meets 5 food rules and 1 or no shopping rules.

Most preferably, objective weighting at step 148 is based on one or more predetermined rules or criterion that assesses the importance of various nutritional attributes of a food items compared to other nutritional attributes and/or other food related attributes based on medical research and scientific data. For example, a rule related to the amount of fat or sodium in a food item may be weighted compared to a rule related to the food item being organic or non-GMO such that fat or sodium level takes precedence over whether the item is organic or non-GMO. As a specific example, if two items having the same total fat level are compared, but one is organic and the other is not and a user weighting rule 116 includes a preference for organic items, the non-organic item may be excluded from a list of items 159 or 162 displayed to the user at step 24 (or may appear at a lower ranking on the list compared to the organic item, depending on the specific rules applied and without applying weighting rule 148). However, if the organic item has more saturated fat per serving than the non-organic item, a rule implemented in step 148 may result in the non-organic item being included on compiled list 159 or 162 (and ultimately displayed to the user at 24) and the organic item not being on compiled list 159 or 162 (and not displayed to the user), or being ranked lower on the list than the non-organic item, based on their respective saturated fat contents (even though the user indicated a preference for organic items at steps 94 and 100 to be used for subjective weighting at step 116). This is also an example of how an objective weighting rule at step 148 may take precedence over a subjective weighting rule at step 116. As another example, dietary and food allergens would also preferably take precedence over food attributes and user preferences (such as non-GMO or vegan) and health goals. Items that do not meet the rules may be omitted from a list displayed to the user or optionally displayed along with a notation that they do not meet the user's criteria and/or objective rules, optionally with explanation as to why the item does not meet the criteria or rules and optionally displayed lower on a list of items that do meet the criteria or rules.

Similar rules may be implemented for objective weighting of shopping rules 156. Objective weighting of shopping rules 156 may also be used to implement sales promotions and/or reduce inventory for particular items as desired by a particular grocer or store with which system 10 is integrated. Any number of objective weighting or ranking factors may be used at steps 148 and/or 156, and various valuations or calculations may be applied to various attributes of a food item to weight or rank that attribute higher or lower than another attribute or preference to compile a list of food items 159 or 162, that are preferably in rank order, as will be understood by one of ordinary skill in the art.

Items failing the comparison/rule application steps in stages 22 and 22A are preferably excluded from the lists at steps 146 and 154, but may be sent to step 153 for further consideration. At optional step 153, a secondary set of objective weighting rules is preferably compared/applied to the failing items, so that at least some of the failing items (most preferably only that that meet, match, or pass at least some of the applied rules) may ultimately be included in list 159 or 162 that is displayed to a user at step 24. This allows for a broader range of food items to be displayed and allows for a user to learn which product and why that product is out of range. For example, an item that has been previously purchased by the user prior to adding in a health goal, allergy or other rule may be displayed and indicated it is now out of range of food preferences and rules. Most preferably, items that fail all applied rules are not sent to optional step 153, but are completely excluded from list 159 or 162. Optional step 153 may be applied, for example, when list 159 or 162 has fewer than a predetermined number of items. Most preferably, when failing items are included in list 159 or 162 under operation of step 153, they are ranked lower than the passing items (appearing lower on the display list), are listed separately or without ranking (such as by alphabetical order or by UPC order), and/or include and indicator or flag (such as a star symbol appearing next to such items) to inform the user that the item does not meet one or more of the applied rules or user's criteria.

Food scores for the items on list 159 are calculated at step 160 (or are retrieved from step 142). Any known algorithms or scoring methods may be used to calculate a food score at step 160, based on nutrition information or other food information retrieved at steps 134 and/or 138. Food scores are objective based on information for the particular food items and are not related to any user specific survey data 78. Food scores may be a numeric value or a non-numeric value on a given scale (such as a star rating out of five stars, color rating, and/or a letter grade selected from A, B, C, D, and E). Optionally, the list compiled at step 159 may be re-ranked or re-ordered at step 162 based on the food scores for the items, with higher food scores being listed before lower food scores and preferably with food items with the same food score maintaining their relative rank as listed in step 159. One or more objective weighting rules 148 may also optionally be applied in step 162 to resolve any conflicts between rankings in list 159 and the food scores to re-rank the items at step 162. Step 22/22A has been primarily described with respect to individual food items, but also applies to recipes (which are preferably assessed according to nutrition information available for the overall recipe per serving, but may also be assessed according to nutrition information and amounts of individual ingredients in a recipe serving).

Referring to FIG. 2B, after comparison and scoring stage 22 (or 22A), a list of items or recipes is displayed at step 24, most preferably in rank order according to one or more of the rules 106, 108, 110, 112, 114, 116, 120, 148, 156, and/or food scores 142 or 160 (user's food criteria rules, shopping criteria rules, subjective weighting/ranking rules, objective weighting/ranking rules, and/or food score, as previously described). Most preferably, food scores from step 142 or 160 are displayed with the items to give the user an easily understandable and objective assessment of the healthfulness of the food item. If recipes are displayed, then the user would select a desired recipe at step 28. Optionally, but preferably, the ingredients necessary for the recipe can be automatically added to the user's cart or shopping list at step 30. The user may also optionally enter a desired number of servings for the recipe and the necessary quantity of ingredients for that number of servings is automatically added to the user's cart or shopping list at step 30. If food items are displayed, the user may select a food item and a quantity to add to the user's cart or list at step 26. A default quantity is preferably set to 1, but may be changed by the user. Most preferably, when an item from the list displayed is selected by a user, specific information regarding that item is also displayed. Such specific information preferably includes a primary food attribute information retrieved at step 134, such as a nutrition facts label and optionally a list of ingredients, as would be displayed on the physical label for the item. A user may optionally select to have additional information regarding the food displayed at step 62. Such additional information may include information retrieved at step 138, such as secondary or tertiary food attributes (similar to steps 402, 404 in system 310), high level attributes (similar to steps 406, 408 in system 310), a “food story” (which preferably includes information on the farmer or manufacturer who produced a particular food item), “fun facts” (such as the number of days it takes from planting to harvest for a particular vegetable) regarding food items, and/or trivia questions related to food items, health, medical conditions, and/or food production/manufacturing, and scientific levels of evidence. Correct answers by a user to trivia questions may also be used to provide a reward to a user in reward stage 34. The information displayed at step 62 may also include food “kudos” (although other terminology may be used), which are certifications, awards, or other forms of recognition received by a food item. For example, a food item may be a certified organic item and that information (or an icon or badge indicating such information) may be displayed at step 62. As an alternative, optional additional information 62 may be automatically displayed to a user or a user may elect to have such information displayed or not as part of the user's survey data 78.

As another preferred embodiment, if food items are displayed, system 10 will also display one or more recipes that use one or more of the foods items displayed. Most preferably, the optionally displayed recipes have been processed through comparison/scoring stage 22/22A and meet one or more of the user's food criteria, shopping criteria, subjective weighting/ranking, objective weighting/ranking, and/or food score, as previously described. A user may select such a displayed recipe and add items to the cart/list in the same manner as described with steps 28 and 30.

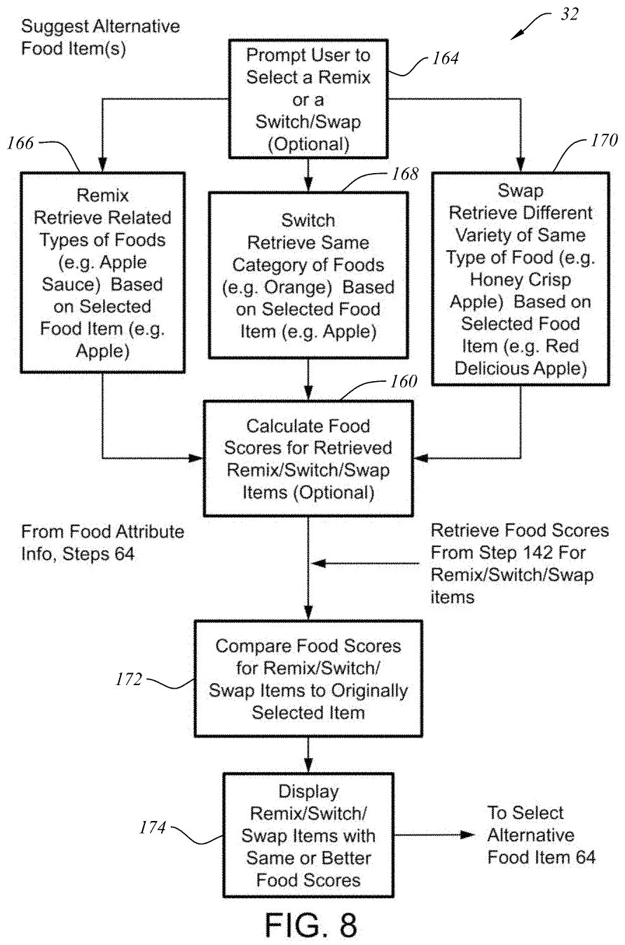

Referring to FIGS. 2B and 8, system 10 also preferably comprises a suggest alternative food items stage 32 to suggest alternatives to the originally list items 159 or 162 or originally displayed items 24. A user may be prompted at step 32 as to whether or not the user wants to view suggested alternative food items and, if so, the user is optionally prompted as to whether the user wants to view “rem ix” items, “switch” items, or “swap” items at step 164. Alternative food items, whether rem ix or switch or swap, may also be automatically suggested. A user may also indicate a preference for suggesting alternatives or not receiving suggested alternatives as part of a user's survey data 78, particularly shopping preferences 96, so that a rule 118 or 124 is carried out to automatically suggest alternative items or to not display suggested alternative items.

A remix item refers to an item that has the same ingredients or similar nutrient mix or range as the original item and is preferably in the same food category e.g. fruits, vegetables, meat, cereal/breakfast, dairy, etc.). For example, if the originally displayed item is an apple, it may remix to apple sauce; or an alternative format of the product, if available. If not available, a remix may display another item of similar taste, satiety, nutrient value, or usage in recipes. A remix item refers to a related, but healthier scored type or category of food based on the originally displayed or selected food. For example, if the originally displayed or selected food is apple juice with added sugar, switch alternatives might include organic apple juice. A switch or swap item refers to the same type of item, but a different variety or a different format. For example, if the originally displayed or selected item is a red delicious apple, then the swap may be a granny smith or honey crisp apple. As another example, if the originally displayed or selected item is canned sweet corn, then the swap item may include fresh corn on the cob or frozen corn. For any given original items, its switch items may overlap with its swap items. Additionally, switch and swap items may be categorized together under a single label (e.g. “switch” or “shuffle”). The use of the terms rem ix, switch, and swap are arbitrary and other terminology, such as “shuffle,” may be used to describe the different types of food alternatives.

If the user selects remix, then remix items are retrieved at step 166. If the user selects switch, then switch items are retrieved at step 168. If the user selects swap, then swap items are retrieved at step 170. A user may optionally select two or more of remix, switch, and swap. Most preferably, a food score is then calculated at step 160 (or retrieved from step 142) for the items retrieved at steps 166, 168, and/or 170. Food scores 142, 160 for the originally displayed items are compared at step 148 to food scores for the alternative remix, switch, swap items from steps 166, 168, and/or 170. Alternative remix, switch, and/or swap items with food scores equal to and/or higher than the food scores for the original items are displayed to a user at step 174.

Optionally, the items retrieved at steps 166, 168, and 170 may enter comparing and scoring stage 22 (or 22A) in the same way originally or initially retrieved items from steps 43, 48, 54, 58, and 60 are compared and scored according to one or more user food criteria rules 118, shopping criteria rules 124, subjective weighting rules 116, objective weighting rules 148, 156, and/or calculated or retrieved food scores 142, 160, rather than only comparing food scores 160, 142 as described above. The overall ranking of the alternative remix, switch, and/or swap items after applying all desired rules and/or food scores used in stages 22, 22A (the list from step 159 or more preferably from step 162 for the remix/switch/swap items) would then be compared to the overall ranking of the original food items after applying all desired rules and/or food scores used in stages 22, 22A (the list from step 159 or more preferably from step 162 for the original items) at step 172. Alternative items of the same and/or better overall ranking compared to the original item would then be displayed to a user as step 174.

Referring to FIG. 2B, a user is preferably prompted at step 64 to select or decline one or more of the alternative suggestions displayed at step 174. If a user does not select an alternative item, then the original item remains in the cart or on the list at step 70. If the user selects one or more of the alternative items at step 66 then the shopping cart or list is preferably automatically updated to replace the original item with the selected alternative item. Most preferably, a reward is displayed to the user or added to the user's profile at step 68. A reward may be given for making a positive food choice by selecting an alternative item better food score and/or overall ranking compared to the original item. A reward may also optionally be given for selecting an alternative item of equal food score and/or overall ranking compared to the original item, or any set achievement which may include reaching an average monthly score increase, adhering to their dietary plan for a duration of time, and other set qualifications.

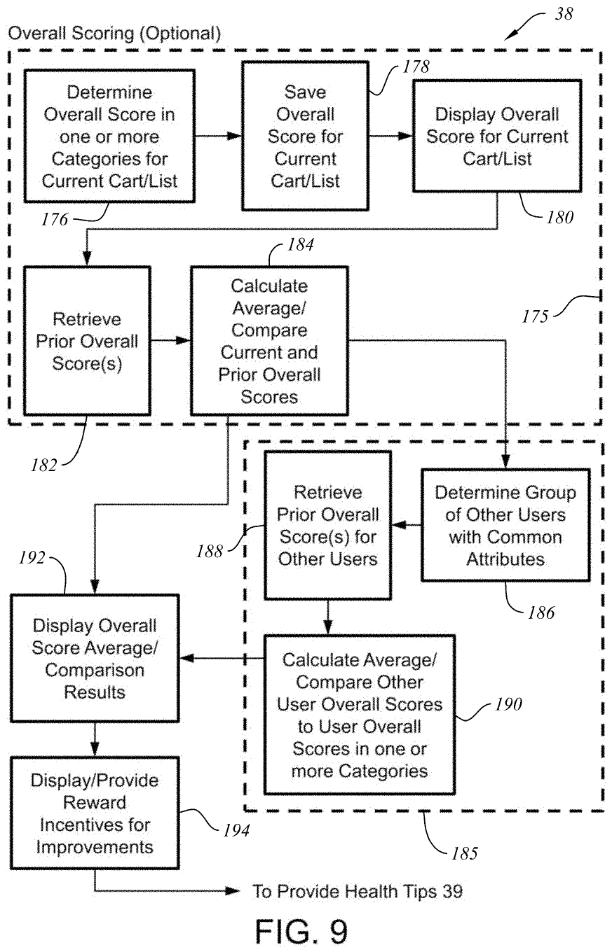

Referring to FIG. 9, system and method 10 also preferably comprises an overall scoring stage 38. Overall scoring stage preferably comprises tracking a user's overall scoring for a particular purchase and historical/previous purchases and providing subjective comparisons to the user's own data and to data from one or more groups of other users to provide a competitive aspect to system and method 10. Any variety of scoring categories may be used in stage 38, including an overall food purchasing (OFP) score (a tally of the food scores for a particular cart/list), the number of better selections made at step 66, and/or the number of particular categories of foods and/or food attributes, etc. For example, a number of vegetables or vegetable servings, and/or total fats, and/or total added sugars, etc. for the current cart/list and historic carts/lists for the first user and the group of users may be compared and displayed. Multiple scoring categories may also be used separately (to display multiple comparisons) or combined in various combinations. Most preferably, a user may select the types of categories that interest the user for use in overall scoring stage 38.

Overall scoring stage 38 preferably comprises determining subjective improvements at step 175 and objective improvements at step 185. Subjective improvements 175 preferably comprises determining overall scores for a scoring category at step 176 for the user's current cart or list (such as a completed cart or list from steps 72 or 74), saving the current overall score for that scoring category at step 178, and displaying the current overall score for that scoring category to a user at step 180. Subjective scoring step 175 also preferably comprises retrieving one or more prior scores for the user in a scoring category at step 182 and calculating an average score for that category from the prior scores or comparing one or more prior scores to the current score at step 184. Objective scoring step 185 preferably comprises comparing attributes from the user's profile information 12 (a first user's profile information) to attributes in a user profile information 12 for one or more other users (such as a second user's profile information, a third user's profile information, etc.) to determine a group of other users with one or more attributes in common with the first user at step 186. Any number and combination of attributes may be used to determine the group of other users at step 186. For example, the other users may reside in the same zip code or metropolitan area as the first user, may have one or more medical conditions in common (such as high blood pressure), may be in the same age range as the first user, same sex, same food preferences (such as vegetarian), adhering to the same health goal (such as diet, lose 10 pounds), etc. Prior scores for any given category for each of the other users (or a representative sample size of such other users) are retrieved at step 188. An average score for the group is preferably determined and compared to the current or average score for the first user in that category at step 190. Alternatively other comparisons may be made between a score for the group and a current or prior score or scores for the first user at step 190 (such as median scores, comparisons of trending scores up or down, etc.). Results of the user's subjective comparisons from step 184 and/or objective comparisons to the group from step 190 are preferably displayed to a user at step 192. Prior scores retrieved at steps 182 and/or 188 may include all prior scores in a given category/categories or be limited in number or by date.

Referring to FIGS. 1, 2B, and 9, reward user stage 34 preferably comprises rewarding a user for making positive selections in stage 32 (at steps 66 and 68); for correctly answering trivia questions regarding health issues, deciding which particular food item is more healthful overall or in a particular category (i.e. which is lowest calorie or lowest fat) than other food items, and/or food production or manufacturing questions; for selecting any original or alternative food item that has a high ranking food score (such as preferably in the top 50% of the scale for the food score, more preferably in the top 60%, and most preferably in the top 80%); for having a total food score or an average food score for a particular cart/list that is above a predetermined threshold; and/or based on one or more positive comparisons in overall scoring stage 38. When a food score is used in assessing rewards, it may be necessary to convert the food score to a numeric value, since some food scores may be in a non-numeric value on a given scale (such as a star rating out of five stars or a letter grade selected from A, B, C, D, and E). In that case, the food score may be converted to a percentage of the overall scale and a reward may be given if the user selects an original food item or an alternative food item that is in the top 50% of the scale or top 60% of the scale, or other predetermined value. For example, on a five-star scale (with five stars being the highest), the top 60% would be a score of 3, 4, or 5 stars. In a five-letter grade scale (with “A” being the highest), the top 60% would be a grade of A, B, or C.

In overall scoring stage 38, a reward is preferably displayed to the user or added to the user's profile at step 194 for improvements in a user's subjective comparisons (e.g. current OFP score compared to the user's own prior OFP scores or total fats in current cart/list compared to total fats in the user's own prior carts/lists) and/or objective comparisons to the group (e.g. user having OFP score(s) higher than scores for the group of other users or user's total fats are lower than total fats for the group of other users). A reward may be provided for making a positive food selection, varying points values may be assigned to food items (such as based on general healthfulness, food score, or subject healthfulness for the specific user) and added to the user's profile when the user selects those food items, or when a user reaches a predetermined milestone or threshold related to a health goal. Rewards stage 34 may also comprise tracking various levels of rewards or points earned 193 and providing a higher reward (such as a higher value coupon or discount) when the next level is achieved. The tracking of points toward a level or goal may also be displayed at step 192 and the higher reward displayed or provided at step 194. A reward at step 68 and/or 194 may be one or more of the following: (1) an alpha-numeric or graphic badge or icon displayed on the user's profile or a user interface screen for system 10; (2) a pop-up graphic (such as shooting stars or a smiley face) that temporarily appears on the user interface screen; (3) points added to a user's profile; and/or (4) discounts or coupons for one or more of the selected alternative items or other food items or merchants (such as restaurants or gyms). If a reward is given as points, the points may be (1) used simply for comparison to other users to evaluate a user's relative performance to users having the same or similar profile characteristics (to provide a sense of competition as an incentive to make better food choices); (2) accumulated and exchanged for one or more promotional or health-related items (such t-shirts, gym memberships, exercise equipment, a FitBit®, etc.); and/or (3) accumulated and exchanged for one or more discounts or coupons for future food item purchases, restaurant purchases, gym memberships, etc.

Rewards stage 34 may also comprise tracking various levels of rewards or points earned 193 and providing a higher reward when the next level is achieved. The tracking of points toward a level or goal may also be displayed at step 192 and the higher reward displayed or provided at step 194.

Referring to FIGS. 1 and 2B, complete shopping/list stage 36 preferably comprises allowing a user to save food items in the cart or on the list for later use at step 72 and may optionally allow for completion of an online purchase of items in the cart at step 74 (if system 10 is integrated with an online shopping platform for a grocer or other store). Step 74 also preferably allows a user to send a list to a printer, another person or user, or another device (e.g. by email or text message), even if system 10 is not integrated with an online shopping platform.

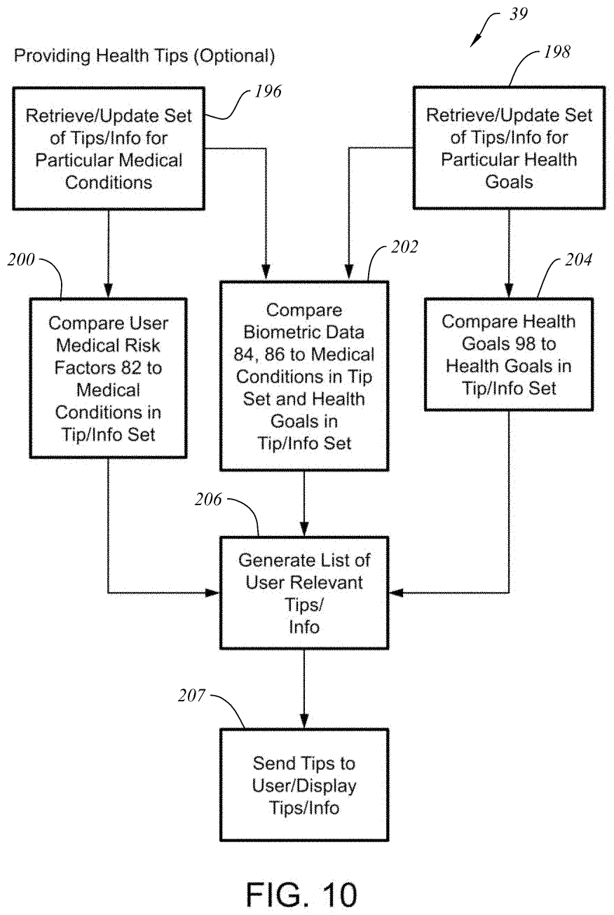

Referring to FIGS. 1 and 10, provide user health tips stage 39 preferably comprises retrieving and periodically updating a set of tips or information related to a particular medical condition at step 196 and a set of tips or information related to particular health goals at step 198. For example, for a high blood pressure medical condition or a health goal of reducing blood pressure, tips or information may include a desired range of “normal” blood pressure readings, steps to reducing blood pressure (such as meditation, reducing stress, and reducing sodium intake), and information regarding blood pressure treatment medications. Information and tips for steps 196 and 198 may also include personal success stories for other persons or users that have improved a particular health condition or met or are on the way to meeting a particular health goal to provide motivation to a user. A user's specific medical risk factors 82, biometric data 84, 86, and health goals 98 are compared to the sets of tips or information at steps 200, 202, and 204 to generate a list or set of user relevant health tips of information at step 206, so that a user receives information specifically relevant to the user's medical conditions, biometric data, and/or health goals. The user relevant tips or information are provided to the user (such as by email, teledoc, telemedicine, nutrition coaching, or text message) or displayed to the user at step 207.

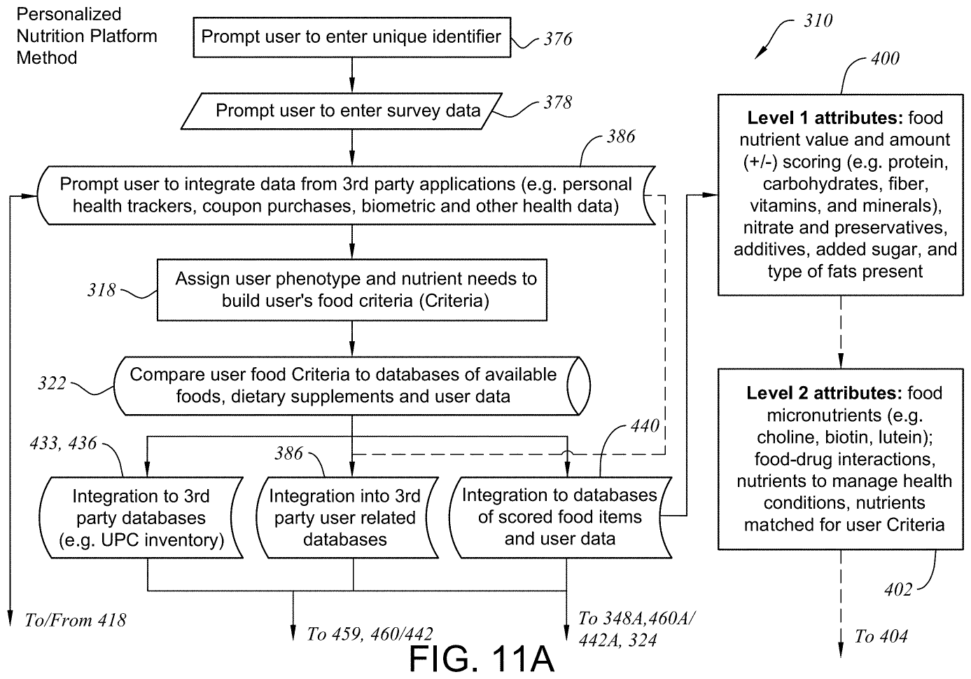

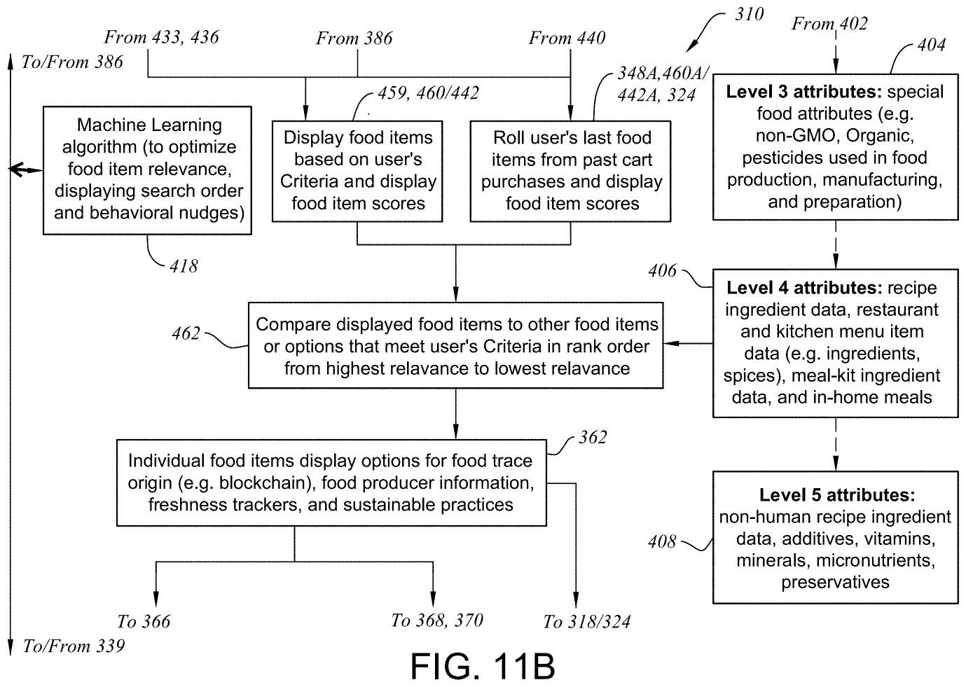

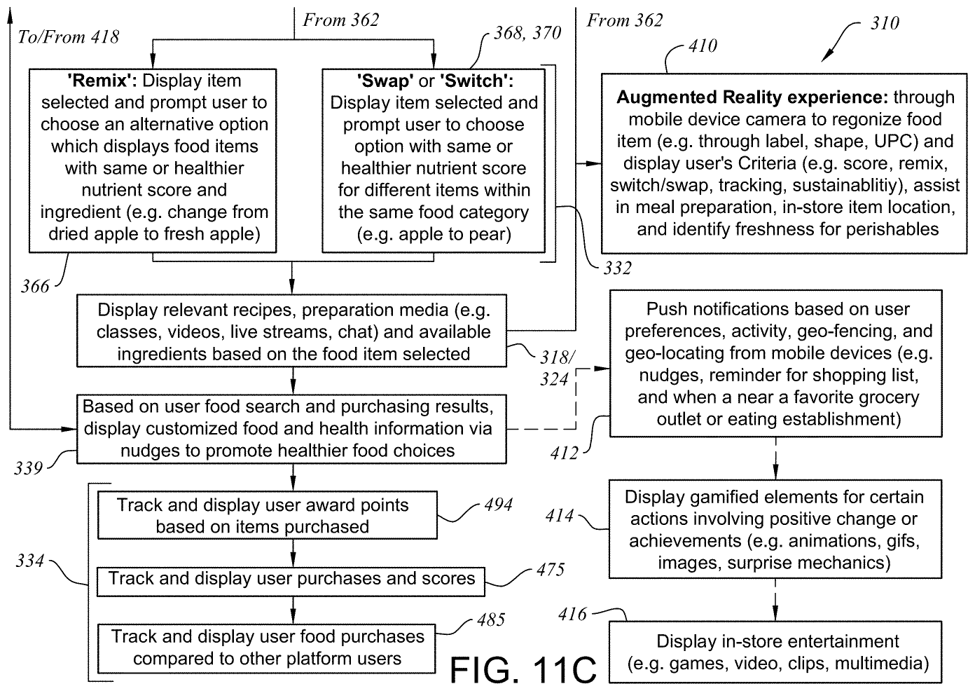

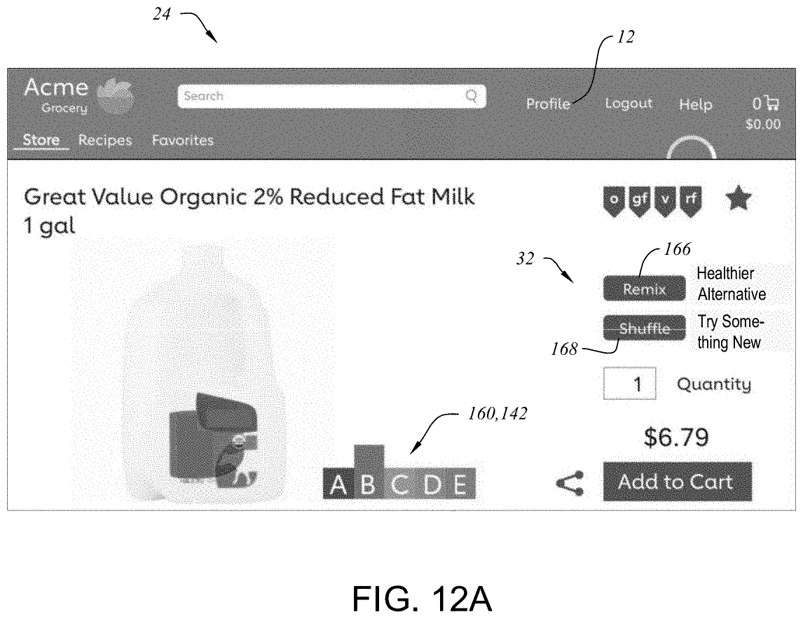

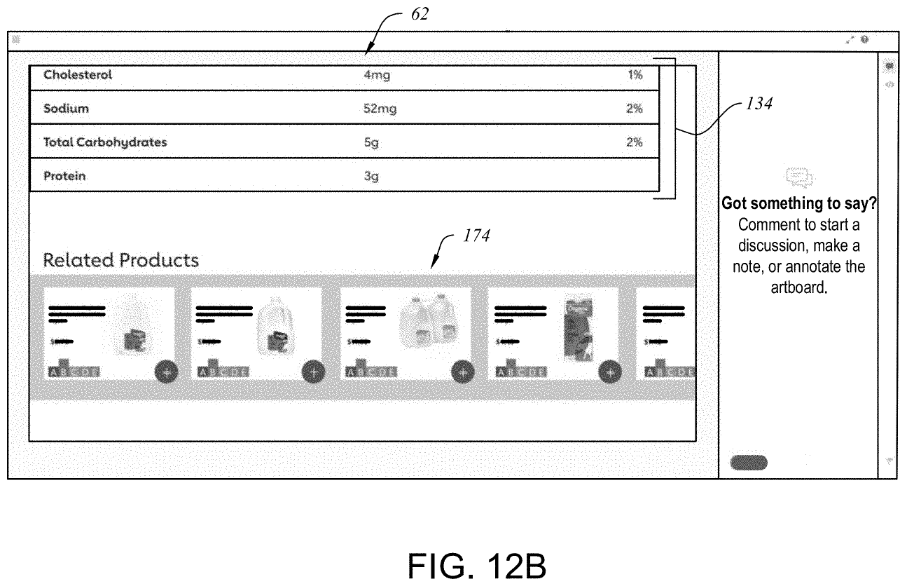

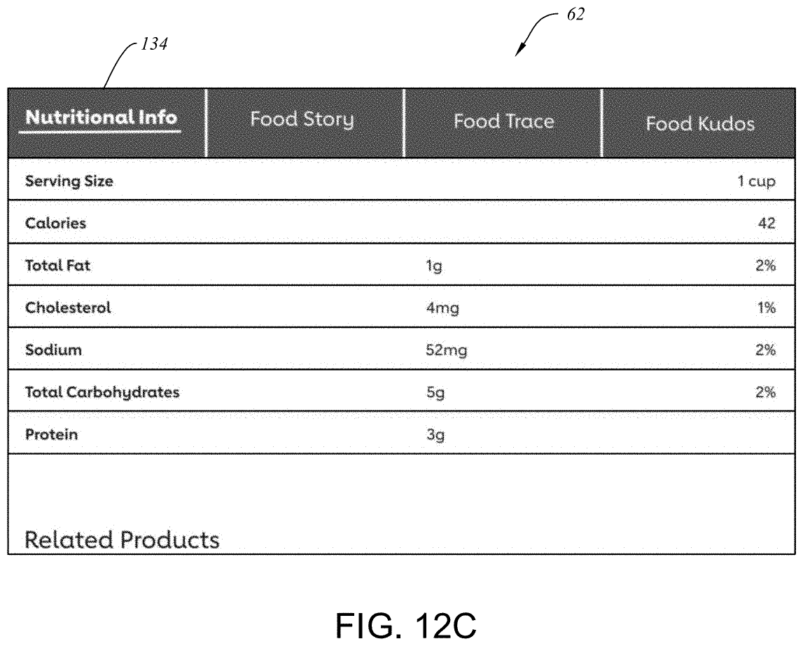

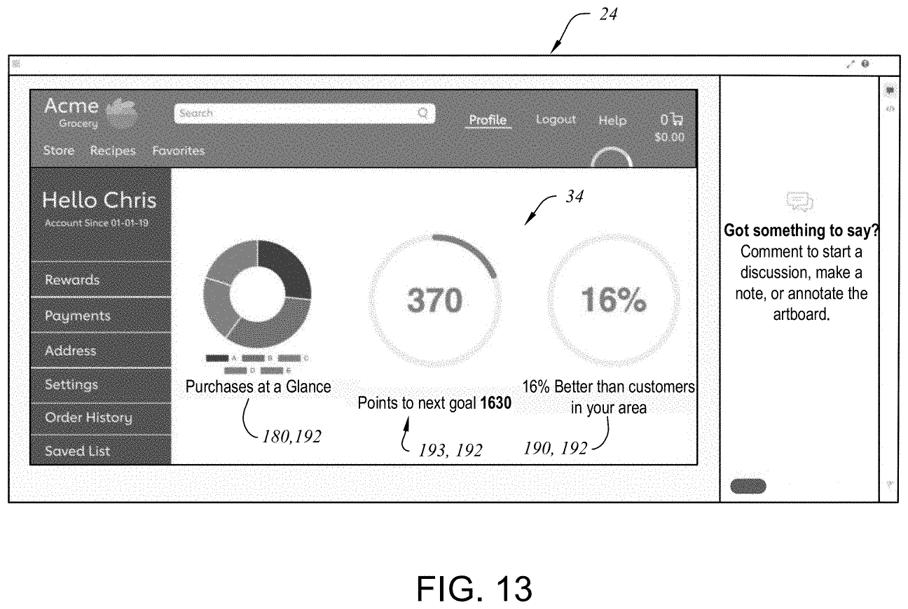

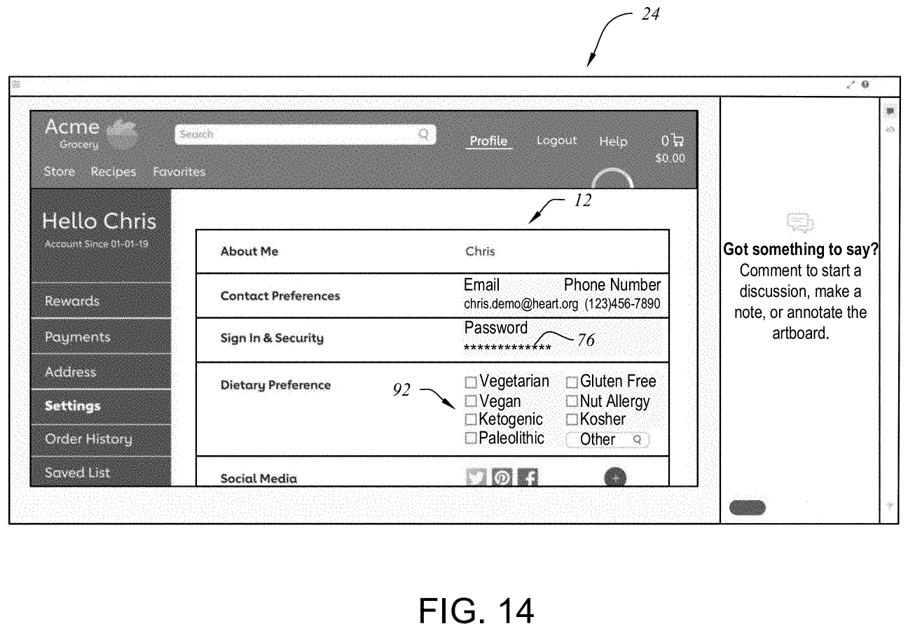

A preferred embodiment of a method and system 310 for improving food selections is shown in FIGS. 11A-11C. System 310 preferably comprises prompting a user to create/enter a login ID and password step 376 and to enter (and optionally update) personal survey data 378. Personal survey data 378 preferably comprises the same information as in step 78 for system 10. A user is prompted to integrate data from third party applications or to link to such applications at step 386. Such third-party applications may include biometric and health data sources, a personal health tracker (such as a FitBit®), and/or coupon collection databases. A user's phenotype and nutrient needs are assigned at step 318 to build a user's food criteria. These are a set of rules based on the user's medical risk factors/medical conditions, biometric data, and/or medications being taken, and may optionally include one or more food preferences, similar to rules 118 in system 10. The user's food criteria are compared to databases of available foods, and optionally dietary supplements and user data at step 322 (similar to stage 22/22A). Integration with various third party systems/databases occurs at steps 386, 432, 436, and 440 (similar to stages/steps 86, 132, 136, 140). Integration with these databases allows level 1 attributes for food items to be retrieved at step 400, level 2 attributes for food items to be retrieved at step 402, level 3 attributes for food items to be retrieved at step 404, level 4 attributes for food items to be retrieved at step 406, and level 5 attributes for food items to be retrieved at step 408. The attributes are later used in comparing food items in step 462. A user's current food items are displayed at steps 459, 460/442, and 324 based on user's criteria and food scores (similar to steps 24, 159 and 160/142 and/or 162). Items from a user's prior purchases are similarly displayed at steps 348A, 460A/442A, and 324 based food scores (similar to steps 24, 48, 160/142, and 162). The food items are then compared and displayed in rank order at step 462 (similar to step 162). Integration with third party systems may also include payment integration (such as PayPal®, Amazon® Pay, Apple Pay®, etc.) to allow a user to complete a purchase.

Referring to FIGS. 11B-11C, additional information regarding food items may be displayed at step 362 (similar to step 62). Alternative suggestions for food items are provided at stage 332 (similar to stage 32), which comprises steps 366 and 368/370 for remix and switch/swap items. Relevant recipes, food preparation media/instructions, and available ingredients are displayed at steps 318 and 324 (similar to stages 18 and 24). Most preferably, an augmented reality step 410 is applied to one or more of steps 362, 332, 318, and 324 as shown on FIGS. 11B-11C. A display of customized food and related health information is displayed at step 339, based on the user's food search and/or prior purchases (similar to stage 39 and the list from step 162). Notifications may be pushed to the user at step 412 (such as through pop-ups, emails, text messages, etc.). User award points based on purchased items are tracked at stage 334 (similar to stage 34). Stage 334 preferably comprises tracking and displaying user award points for purchased items at step 494 (similar to step 194), tracking and displaying user purchases and scores at step 475 (similar to step 175), and tracking and displaying user's purchases as compared to other users at step 485 (similar to step 185).

Most preferably, machine learning algorithms 418 are applied to one or more of the steps as indicated in FIGS. 11A-11C, to allow system 310 to apply iterative learning and artificial intelligence techniques to enhance and optimize results. Gameified elements are preferably displayed at step 414, such as animations, gifs, or images that reward certain actions by user, such as positive food choices. In-store entertainment is preferably displayed at step 416.

Systems 10 and 310 may also be used in connection with prepackaged food delivery services and restaurants, in the same or similar manner as described herein for grocery items, to provide a user with information regarding various food items, menu items, alternative foods items, and to provide incentives for making healthy food choices.

According to one preferred embodiment, a system for carrying out the preferred method of improving food selections according to the invention comprises:
one or more computers or terminals or other computing devices, with or without a server, accessible by a user, to allow data entry in response to prompts regarding personal information and risk factors; to provide or display information, lists, scores, rewards, and other items discussed above with respect to system and method 10 and/or 310; optionally to receive or allow for printing or download of health information and/or food attribute and food information; and/or optionally to allow communication via the internet with one or more external sites (an external computer, terminal, or computing device, an external database, an externally hosted software site, file sharing site, and/or a healthcare provider computer, terminal, or computing device);
a software component at the user's computer, terminal, computing device, or server or remotely accessible from such (hosted at the software provider's or other third party's site) for managing, initiating, and carrying out the method steps of the personal survey data entry, calculations and comparison, integration with databases, and optional provision of health information and rewards/scoring tracking, and optionally configured to integrate with one or more online shopping, payment processing, or ordering platforms to allow food items to be purchased from a grocer, store, restaurant, food delivery service, or other merchant;
a database system or similar data storage capability for storing data entered as personal survey data, user criteria rules to be applied, optional subjective and/or objective weighting rules, optional health information or tips, and/or optional historic scoring, rewards, and purchasing records the database system accessible by the software component or integrated into the software component;
an optional database system or similar data storage capability for storing food attributes and information and food scores, the optional database system accessible by the software component or integrated into the software component;
if the software component is hosted externally from the user's device, a computerized server connected to a network to host the software, preferably containing the database system and optional database systems, and to allow communication with and between one or more user computers, terminals, or other computing devices; one or more users/patients, including by email; and optionally one or more external sites (an external database, file sharing site, or a remote healthcare provider or patient computer or terminal) via the internet.

Computer, terminals, and computing devices preferably have a processor and memory storage capability to allow execution of the software component to carry out the steps of the methods according to the invention. Various embodiments of a method according to the invention may also be carried out on a computer, tablet, smart phone, stand-alone kiosk, or similar device having a user interface to allow input of a patient's data, a processor for making calculations, and the ability to store data.

One or more components or steps described with one preferred embodiment of a system or method according to the invention may be used with another embodiment of the system or method, even if not specifically described in connection with the embodiment. Reference numerals used to describe steps of a method herein may also describe the information, data, or results that are input and/or output from such step. For example, reference number 12 refers to user profile entry step and refers to the profile information entered at that step and reference number 48 refers to retrieving previous cart/list step and refers to the food items retrieved at that step. Although not specifically depicted in the figures, additional steps may be added, including additional steps to save data, information, rules, rewards, or results, as will be understood by those of ordinary skill in the art. The shapes used in the flow chart figures are not intended to be limiting to a particular type of flow chart step. References to servers, computers, terminals, and other computing devices are intended to include all such devices that permit the data entry, communication, storage, and access to data and information, and/or that are capable of carrying out steps defined in a software program. Steps described herein as a prompt to a user may alternatively be a tab, drop-down box, or other option to be selected by the user or recipient. Unless a specific format is indicated as required, references herein to displaying or providing results or information include any manner of display or providing the results or information, such as displayed on a screen, printouts, postal mail, courier, sent by email, providing access to downloadable information, providing link to external websites containing information, displaying graphics, displaying photos, and displaying videos, providing pop-up blocks containing information, photos, videos, or graphics, uploaded to a file sharing site, text, or other electronic means. Any combination of optional stages or steps may be used in connection with the preferred methods and system of the invention. Those of ordinary skill in the art will also appreciate upon reading this specification, that modifications and alterations to the methodology and system for improving food purchases may be made within the scope of the invention and it is intended that the scope of the invention disclosed herein be limited only by the broadest interpretation of the appended claims to which the inventors are legally entitled.

What is claimed is:

1. A method for improving healthfulness of a user's food selections based on one or more health factors, the method comprising:
obtaining information regarding the one or more health factors specific to the user;
obtaining information regarding one or more first food items;
calculating or retrieving a food score for each of the first food items;
generating a first food list;
displaying the first food list to the user; and
wherein the first food list comprises the one or more first food items and the food score for each first food item.

2. The method of claim 1 further comprising:
creating or retrieving one or more general health rules for the one or more health factors;
creating or retrieving one or more user specific health rules based on the general health rules and the one or more health factors specific to the user;
applying the one or more user specific health rules to each of the first food items;
determining a match score for each first food item relative to the user specific health rules;
wherein the first food list comprises the one or more first food items ranked in order of the (1) the food score for the item, (2) the match score for the item, or (3) a combination of food score and match score for the item.

3. The method of claim 2 wherein the one or more health factors comprise one or more of hypertension, blood pressure measurement above a predetermined threshold for blood pressure, lipids above a predetermined threshold for lipids, diabetes, cholesterol above a predetermined threshold for cholesterol, BMI, weight, food allergy, and food sensitivity.

4. The method of claim 2 further comprising:
obtaining information about one or more alternative food items;
calculating or retrieving a food score for each of the alternative food items;
generating a second food list;
displaying the second food list to the user; and
wherein the second food list comprises the one or more alternative food items that have a food score equal to or higher than at least one of the first food items.

5. The method of claim 4 further comprising:
applying the one or more user specific health rules to each of the alternative food items;
determining a match score for each alternative food item relative to the user specific health rules; and
ranking the alternative items on the second food list in order of the (1) the food score for the item, (2) the match score for the item, or (3) a combination of food score and match score for the item.

6. The method of claim 5 further comprising:
allowing the user to select or decline one or more of the alternative food items on the second list, wherein selecting the alternative food item orders the item, places the item in a cart for purchase, or places the item on a shopping list;
providing the user with a reward for selecting one or more of the alternative food items on the second list.

7. The method of claim 1 further comprising:
allowing the user to select or decline one or more of the first food items on the first list, wherein selecting the first food item orders the item, places the item in a cart for purchase, or places the item on a shopping list;
optionally assigning a point value to each of the first food items; and
providing the user with a reward (1) for selecting one or more of the first food items that is that meets one or more predetermined criteria, (2) reaching a predetermined threshold for total points associated with each of the first food items selected by the user; (3) reaching a predetermined milestone in meeting a health goal of the user, or (4) a combination thereof.

8. The method of claim 1 further comprising:
allowing the user to select or decline one or more of the first food items on the first list, wherein selecting the first food item orders the item, places the item in a cart for purchase, or places the item on a list to create a shopping list;
determining an overall score for all selected first food items;
tracking overall scores for the user's selections over multiple orders, purchases, or created shopping lists; and
comparing the overall scores for a current order, purchase, or created shopping list to the overall scores for one or more prior orders, purchases, or created shopping lists; and
(1) displaying the comparison to the user or (2) providing a reward to the user if the comparison shows improving overall scores or (3) both.

9. The method of claim 8 further comprising:
determining a group of other users having one or more attributes in common with the user and repeating all steps for each other user;
periodically determining an overall average score for the group for all selected first food items for each order, purchase, or created list by all users in the group over a period of time;
comparing the overall average score for the group to (1) one or more of the user's overall scores or (2) optionally an average overall score for the user over the period of time; and
(1) displaying the comparison to the user or group or both, or (2) providing a reward to the user if the comparison shows the user's overall score is better than or improving compared to the overall average score of the group or (3) both.

10. The method of claim 1 further comprising:
obtaining one or more food preferences for the user;
comparing the information regarding the first food items to the one or more food preferences for the user;
determining a match score for each first food item relative to the one or more food preferences for the user;
wherein the first food list comprises the one or more first food items with match scores above a predetermined threshold and the food score for each of those first food items.

11. The method of claim 10 further comprising:
displaying information related to the one or more food preferences for the user for each of the first food items on the first food list; and
wherein the one or more food preferences comprise one of more of the following related to food items: vegetarian, vegan, non-GMO, locally produced, sustainability of production or processing, source, diary free, sugar free, no added sugar, gluten free, preservative free, no added food colors, or no high fructose corn syrup.

12. The method of claim 11 wherein the first food list comprises the one or more first food items ranked in order of the (1) the food score for the item, (2) the match score for the item, or (3) a combination of food score and match score for the item.

13. The method of claim 1 further comprising:
allowing the user to search for the one or more first food items;
displaying the information regarding the one or more first food items to the user;
and wherein the information comprises: (1) nutritional information, (2) ingredient information, (3) whether the first food items are vegetarian, vegan, non-GMO, locally produced, diary free, sugar free, no added sugar, gluten free, preservative free, no added food colors, no high fructose corn syrup, or any combination thereof, (4) food production and packaging information, or (5) any combination thereof.

14. The method of claim 13 wherein the food production and packaging information comprises sustainability information, where the food item was grown or processed, environmentally friendly aspects of food production, environmentally friendly aspects of packaging, type of packaging, information regarding the farmer or manufacturer, or any combination thereof.

15. The method of claim 2 wherein the one or more health factors specific to the user comprise a health goal, the method further comprising: providing information to the user regarding (1) the one or more health goals or (2) one or more food items related to the one or more health goals.

16. The method of claim 15 further comprising periodically obtaining biometric data related to the one or more health goals and displaying the user's progress in meeting the one or more health goals.

17. The method of claim 3 further comprising:
obtaining information about one or more alternative food items;
calculating or retrieving a food score for each of the alternative food items;
generating a second food list comprising the one or more alternative food items, wherein the food score of each alternative food item is equal to or higher than at least one of the first food items;
applying the one or more user specific health rules to each of the alternative food items;
determining a match score for each alternative food item relative to the user specific health rules;
ranking the alternative items on the second food list in order of the (1) the food score for the item, (2) the match score for the item, or (3) a combination of food score and match score for the item;
displaying the second food list to the user with the ranking or in a ranked order;
allowing the user to select or decline (1) one or more of the first food items on the first list, (2) one or more of the alternative food items on the second list, or (3) a combination thereof, wherein selecting the first food item or alternative food item orders the selected item, places the selected item in a cart for purchase, or places the selected item on a shopping list;
providing the user with a first reward for selecting one or more of the alternative food items on the second list.
determining an overall score for all selected items for each order, purchase, or created shopping list;
tracking overall scores for the user's selections over multiple orders, purchases, or created shopping lists; and
comparing the overall scores for a current order, purchase, or created shopping list to the overall scores for one or more prior orders, purchases, or created shopping lists; and
(1) displaying the comparison to the user or (2) providing a second reward to the user if the comparison shows improving overall scores or (3) both.

18. The method of claim 1 further comprising:
obtaining user specific information regarding one or more (1) food preferences for the user, (2) shopping preferences for the user, (3) food allergies for the user, (4) food sensitivities for the user, (5) health goals for the user, or (6) a combination thereof;
creating or retrieving one or more user specific preference rules based on the user specific information;
allowing the user to assign a weight to one or more of the user specific information based on relative importance to the user;
creating or retrieving one or more subjective weighting rules based on the user assigned weight of the one or more user specific information;
creating or retrieving one or more user specific health rules based on the one or more health factors specific to the user;
wherein the generating the first food list step comprises: (1) applying rules to each of the first food items, the rules comprising (a) the user specific preference rules, (b) the subjective weighting rules, and (c) the user specific health rules; and (2) ranking each of the first food items according to (a) how well the item meets the applied rules, (b) the food score of item, or (c) a combination thereof;
obtaining information regarding one or more alternative food items corresponding to at least one of the first food items by being (1) in the same food category as the at least one of the first food items or (2) the same type of food as the at least one of the first food items, but a different variety or in a different format;
calculating or retrieving a food score for each of the alternative food items;
generating a second food list comprising each alternative food item having its food score equal to or higher than the food score of its corresponding first food item;
displaying the second food list to the user;
allowing the user to select or decline one or more of the items on the second food list and the first food list, wherein selecting the item orders the selected item, places the selected item in a cart for purchase, or places the selected item on a shopping list; and
wherein the one or more food preferences comprise one of more of the following related to food items: vegetarian, vegan, non-GMO, locally produced, sustainability of production or processing, source, diary free, sugar free, no added sugar, gluten free, preservative free, no added food colors, or no high fructose corn syrup.

19. The method of claim 18 wherein the applying rules step further comprises weighting the user specific health rules over the user specific preference rules.

20. The method of claim 19 further comprising:
determining an overall score for all selected items for each order, purchase, or created shopping list;
tracking historic overall scores for the user's selections over multiple orders, purchases, or created shopping lists; and
comparing the overall score for a current order, purchase, or created shopping list to the overall scores for one or more prior orders, purchases, or created shopping lists;
determining a group of other users having one or more attributes in common with the user and repeating all steps for each other user;
periodically determining an overall average score for the group for all selected first food items for each order, purchase, or created list by all users in the group over a period of time;
comparing the overall average score for the group to (1) one or more of the user's overall scores or (2) optionally an average overall score for the user over the period of time;
displaying the comparisons to the user; and
providing a reward to the user if either comparison shows the user's overall score is better than or improving compared to the user's historic overall scores or the group's average overall score.
